(19) United States
(12) Patent Application Publication
(10) Pub. No.: US 2025/0129076 A1
(43) Pub. Date: Apr. 24, 2025

(54) FUSED TETRACYCLIC COMPOUNDS, COMPOSITIONS AND DIAGNOSTIC APPLICATIONS THEREOF

(71) Applicant: Fathima Benazir, Bengaluru (IN)

(72) Inventors: Fathima Benazir, Bengaluru (IN); Nadim Shaikh, Pune (IN)

(21) Appl. No.: 18/703,379

(22) PCT Filed: Aug. 12, 2022
(86) PCT No.: PCT/IN2022/050731

§ 371 Date: Apr. 22, 2024

(30) Foreign Application Priority Data
Aug. 12, 2021 (IN) 202141036582

(51) Int. Cl. C07D 471/22; C07C 39/17; C07D 491/22; C07D 493/22; C07D 495/22; C09K 11/06

(52) U.S. Cl. CPC C07D 471/22; C07C 39/17; C07D 491/22; C07D 493/22; C07D 495/22; C09K 11/06

(57) ABSTRACT

The present invention discloses methods of preparation of fluorescent nucleic acid staining agents containing fused tetracyclic, heterocyclic compounds of formula (I), their tautomers, polymorphs, stereoisomers, solvates, and their applications in the detection of nucleic acids, and assay methods to establish the efficacy of the nucleic acid staining agents as given in formula (I)

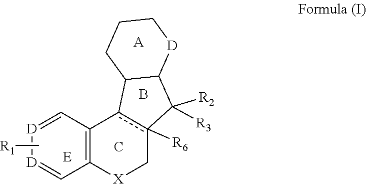

### TECHNICAL FIELD OF THE INVENTION

The invention discloses the process of preparation of fused tetracyclic, heterocyclic compounds, and pharmaceutical compositions containing them for application in the detection of nucleic acids as staining agents.

### BACKGROUND OF THE INVENTION

Fused tetracyclic, heterocyclic compounds with isoquinoline nucleus find extensive application as pharmaceutical compositions for treating a variety of disorders effectively (Ramadan A. Mekheimer et al., Advancements in the synthesis of fused tetracyclic quinoline derivatives, RSC Advances, Issue 34, 2020). They are very good candidates in the treatment of cancer owing to their binding with the DNA of the targeted cells (Tsung-ChihChen et al., Design, synthesis and biological evaluation of tetracyclic azafluorenone derivatives with topoisomerase I inhibitory properties as potential anticancer agents, Arabian Journal of Chemistry, Volume 12, Issue 8, December 2019, Pages 4348-4364).

They can as well form an interesting class of staining agents for detecting nucleic acids, due to their tendency to bind with the DNA and RNA owing to the moieties or substituents present in the rings. The reactant moieties present on the cyclic structures can bind with the phosphate or the sugar groups of the DNA forming fluorescent compounds, which can be detected by a suitable detecting mechanism (U.S. Pat. No. 5,401,847A). This property of the fused-tetracyclic and heterocyclic compounds (Rupesh Nanjunda and W. David Wilson, Binding to the DNA Minor Groove by Heterocyclic Dications: From AT Specific Monomers to GC Recognition with Dimers, Curr Protoc Nucleic Acid Chem. 2012 December; CHAPTER: Unit 8.8) makes them eligible as marker reagents for identifying the nucleic acids from various cells, cell organelles, tissues, smears, biological liquids, as well in diagnostic, curative and forensic applications.

Ethidium Bromide, the versatile nucleic acid staining agent when it was invented became the choice for the detection of DNA at once, from a variety of cell organelles. Since it brings about undesirable changes in the nucleic acids leading to mutagenicity, and genotoxicity, it is replaced subsequently by many fluorescent staining agents such as SYBR, orange Red, SYBR-Green, SYBR-safe, xylenol cynol, bromophenol blue, gel red, DAPI etc. The most desirable quality of a nucleic acid staining agent would be its ability to permeate through the cell membrane and bind with the nucleic acid to produce sensitive and accurate results through gel electrophoresis and their quantification techniques such as PCR, RT PCR, flow cytometry and other spectrofluorimetric quantification assays.

The staining agents can be intercalating or non-intercalating depending on their interaction mechanism with the nucleic acid strands. They can form an inner groove complex or external groove complex with the DNA base pairs. If its mechanism is an external groove binding, it is safe for the specimen and devoid of genotoxicity as it is not tampering with the structure of the nucleic acid. For example, SYBR Green is a green fluorescent cyanine dye that has high affinity for double-stranded DNA. The mode of binding is believed to be a combination of DNA intercalation and external binding. When bound, SYBR absorbs at a wavelength around 497 nm and emits fluorescence around 530 nm. Presently SYBR and similar staining agents are finding extensive applications in DNA testing and analysis.

Though SYBR is safe and sensitive compared to ethidium bromide, it is not very sensitive and many times false positive results are obtained. Since it is a double strand specific (dsDNA) staining agent it is not suitable for single strand DNA detection, RNA detection. Safer stains do exhibit variations in cost, sensitivity and the impedance of DNA as it migrates through the gel. Intercalating dyes change the charge and flexibility of DNA molecules and add to the weight, altering the movement of the dye-nucleic acid complex through the gel when employed as a preloading agent. The post-staining method is therefore the most accurate way to size DNA fragments, but it is time-consuming and costly. Nucleic acid stains that use considerably less stain than standard protocols, have high affinities for nucleic acids exhibit very high fluorescence enhancements upon binding (up to 1000-fold) compared to conventional stains such as ethidium bromide are the preferred ones.

Another important criterion is that the stain should penetrate through thick gels easily for fast and even staining. It should be sensitive for dsDNA, ssDNA and RNA using a standard 300 nm UV transilluminator, enabling one to obtain high sensitivity without using expensive laser scanners.

The primary disadvantages associated with dyes such as SYBR are generating false positive signals, lack of sensitivity, and toxicity (though not to the extent of ethidium bromide). Since SYBR dye binds to any double-stranded DNA, it can also bind to nonspecific double-stranded DNA sequences. Therefore, it is extremely important to have well-designed staining agents that are non-toxic and do not amplify non-target sequences.

Keeping these aspects in mind the present invention is taken up to overcome the inadequacies experienced by the currently used nucleic acid staining agents, and come out with staining agents that can bind to dsDNA, ssDNA, RNA, DNA-bound protein molecules, cell organelles (patent CA2734273A1). In furtherance the nucleic acid staining agent should be able to give specific and sensitive measurements, not restricting the mobility of the DNA on binding through the gel, compatible with detecting techniques such as real-time PCR etc. It should ensure less toxic waste disposal after measurements, less toxicity and mutagenicity on binding with the nucleic acid, non-intercalating, and cost-effectiveness.

The present invention ensures faster, accurate, sensitive and safe detection of nucleic acids, and at the same time finds application in pharmaceutical compositions owing to the presence of fused tetracyclic, heterocyclic nucleus with target-specific moieties present in it.

Prior art showed some similar inventions having applications as staining agents.

Patent WO2001086264A1 disclosed dyes suited for staining of nucleic acids, particularly suitable for staining of RNA in reticulocytes, staining DNA in nucleated red blood cells and compositions and methods for facilitating rapid transport of dye molecules through a cell membrane consisting of at least one surfactant and optionally, a sulfonic acid or a salt thereof.

WO2012040924A1 disclosed compositions comprising at least one fused tetracyclic heterocyclic compound, and methods of using the fused tetracyclic heterocycle compounds for treating or preventing HCV infection in a patient.

Patent EP473563 revealed compounds that can be used to prepare dye conjugates that are uniformly and substantially more fluorescent on proteins, nucleic acids or other biopolymers, than conjugates labeled with structurally similar known carbocyanine dyes.

Patent US20120059001 disclosed 4H-CHROMEN-4-ONE COMPOUNDS AS MODULATORS OF PROTEIN KINASES, methods of preparing them, pharmaceutical compositions containing them and methods of treatment, prevention and/or amelioration of kinase mediated diseases or disorders with them.

U.S. Pat. No. 5,401,847A disclosed heteromultimeric fluorophores for binding to DNA, which allow for the detection of DNA in electrical separations and preparation of probes having high-fluorescent efficiencies and large Stokes shifts.

CA2734273A1 provided compounds, methods and kits for identifying in cells of interest organelles including nuclei and a wide variety of organelles other than nuclei (non-nuclear organelles), as well as cell regions or cell domains. CA2734273A1 disclosed the preparation and use of fluorescent dyes comprising polycyclic fused ring systems, such as anthraquinone, anthrapyrazole, and benzophenoxazine fluorophores as well as their aza derivatives in cell imaging and detection. Generally, these types of dyes are electrically neutral and lipophilic, properties that permit them to be better solubilized in non-polar environments, such as cell membranes thereby rendering them cell permeable. More particularly, the invention relates to modifications of these dyes with functional groups that target the dyes to various subcellular organelles or regions.

Though the prior cited throws light on various nucleic acid staining agents, there is a void to be filled with highly specific, sensitive, nucleic acid staining agents that can detect single-strand, double-strand DNA, RNA, Proteins, cell-organelles, with no toxicity to be used for direct imaging and monitoring of targeted cells and are cost-effective. The present invention is conceived keeping in mind these aspects for consideration.

The present invention contains fused tetracyclic, heterocyclic nucleic acid staining agents that possess moieties primarily to facilitate binding to nucleic acids and proteins. These are fluorescent indicators, which can detect nucleic acids using UV excitation, or other spectrofluorimetry, real-time PCR, flow cytometry, microscopy and imaging methods of analysis. The objective was to enhance the binding abilities of these molecules to nucleic acids and proteins. Detection apparatus comprised gel documentation system, UV transilluminator or spectrofluorimeter, fluorescence/confocal microscopy, flow-cytometry and RT-PCR.

### SUMMARY OF THE INVENTION

The exemplary aspect of the present invention discloses compounds of formula (I), their tautomers, polymorphs, stereoisomers, solvates, and its applications in the detection of nucleic acids.

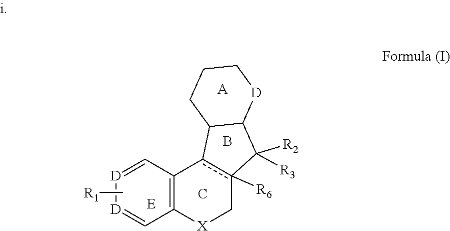

The exemplary embodiment of the present invention disclosed compounds of formula (I) wherein, A is polycyclic heterocyclic ring which is unsaturated or partially unsaturated optionally having up to two heteroatoms independently selected from O, N or S; Ring A can be optionally substituted by the atoms or group selected from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl.

In another embodiment of the present invention of compounds of formula (I) wherein, D is selected from CR⁴ or N;

In another embodiment of the present invention of compounds of formula (I) wherein X is selected from CR⁴, O, NR⁵ or S.

Yet another embodiment of the present invention of compounds of formula (I) wherein, ring C is saturated or partially unsaturated, when it is saturated R6 can be H or OH whereas in case ring C is partially unsaturated R6 is absent.

In another embodiment of the present invention of compounds of formula (I) wherein, R¹ and R⁴ are selected from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl.

In an important aspect of the present invention of compounds of formula (I), R¹ and R⁴ can cyclize to form a 4-7 membered ring which can be optionally substituted by hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl.

In another embodiment of the present invention of compounds of formula (I) wherein R² and R³ are independently selected from hydrogen, alkyl, cycloalkyl alkenyl, alkynyl, alkoxy, acyl, acylamino, optionally R² and R³ can combine to form a 3 to 7 membered ring.

In one preferred embodiment of the present invention of compounds of formula (I) wherein, R⁵ is selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl.

In an exemplary aspect of the present invention of compounds of formula (I), the application of the compounds as nucleic acid staining agents to detect nucleic acids from nucleus and non-nucleus cell organelles and proteins.

Yet another embodiment of the invention of compounds of formula (I) are useful as pharmaceutical compositions.

In one preferred embodiment of the invention of compounds of formula (I) are its tautomers, stereoisomers, solvates, polymorphs, prodrugs, and pharmaceutically acceptable salts.

In one exemplary aspect of the invention of compounds of formula (I), the invention discloses the preparation and application of prophetic molecules numbered 1-34 along with their structures as given in table 1.

Yet another important aspect of the invention of compounds of formula (I) is that the compounds finding application as nucleic acid staining agents are non-intercalating which shows their non-mutagenicity and non-toxic nature.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

### BRIEF DESCRIPTION OF FIGURES

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings.

FIG. 1: Illustrates agarose gel shows different concentrations of RNA stained with compounds, according to the aspects of present invention.

FIG. 2: Illustrates transverse section of plant cell stained with one of the compounds viewed under Olympus confocal microscope, according to the aspects of present invention.

FIG. 3: Illustrates Buccal cell nucleus stained with one of the compounds viewed u see fluorescent microscope, according to the aspects of present invention.

FIG. 4: Illustrates cell division stages stained with one of the compounds and viewed under a confocal microscope, according to the aspects of present invention.

FIG. 5: Illustrating that the compound stains only live yeast cells and not dead cells. The yeast cells were stained without permeabilization and observed under a confocal microscope, according to the aspects of present invention.

FIG. 6: Illustrates yeast nuclei clearly stained and viewed under apotome, according to the aspects of present invention.

FIG. 7: Illustrates plant pollen stained with compound, according to the aspects of present invention.

FIG. 8: Illustrates plant stomata stained, according to the aspects of present invention.

FIG. 9: Illustrates HeLa cells stained with compound and observed under a confocal microscope, according to the aspects of present invention.

FIG. 10: Illustrates PCR products stained and observed in Biorad XRS gel doc system, according to the aspects of present invention.

FIG. 11: Illustrates Plasmid DNA stained with compound and viewed under gel doc system, according to the aspects of present invention.

FIG. 12: Illustrates Compound 7 shows 25 fold increase in fluorescence upon binding with Calf Thymus DNA (CT-DNA), according to the aspects of present invention.

FIG. 13: Illustrates Compound 9 shows 15 fold increase in fluorescence upon binding with Calf Thymus DNA (CT-DNA), according to the aspects of present invention.

FIG. 14: Illustrates Compound 18 shows 70 fold increase in fluorescence upon binding with Calf Thymus DNA (CT-DNA), according to the aspects of present invention.

FIG. 15: Illustrates Compound 16 shows 5 fold increase in fluorescence upon binding with Calf Thymus DNA (CT-DNA), according to the aspects of present invention.

FIG. 16: Illustrates Compound 17 shows >1000 fold increase in fluorescence upon binding with Calf Thymus DNA (CT-DNA), according to the aspects of present invention.

FIG. 17: Illustrates Compound 19 shows >1000 fold increase in fluorescence upon binding with Calf Thymus DNA (CT-DNA), according to the aspects of present invention.

FIG. 18: Illustrates Compound 25 shows >1000 fold increase in fluorescence upon binding with Calf Thymus DNA (CT-DNA), according to the aspects of present invention.

FIG. 19: Illustrates Compound 33 shows >1000 fold increase in fluorescence upon binding with Calf Thymus DNA (CT-DNA), according to the aspects of present invention.

FIG. 20: Illustrates Compound 35 shows >1000 fold increase in fluorescence upon binding with Calf Thymus DNA (CT-DNA), according to the aspects of present invention.

FIG. 21: Illustrates Compound 37 shows >1000 fold increase in fluorescence upon binding with Calf Thymus DNA (CT-DNA), according to the aspects of present invention.

FIG. 22: Illustrates Compound 38 shows 2 fold increase in fluorescence upon binding with Calf Thymus DNA (CT-DNA), according to the aspects of present invention.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

### Fused Tetracyclic Compounds, Compositions and Diagnostic Applications Thereof

### PRIORITY PARAGRAPH/CROSS-REFERENCE TO RELATED APPLICATIONS

This application claims priority to Indian Provisional Patent Application No. 202141036582 filed Aug. 12, 2021 titled “FUSED TETRACYCLIC COMPOUNDS, COMPOSITIONS AND DIAGNOSTIC APPLICATIONS THEREOF,” which is incorporated by reference in its entirety.

It is to be understood that the present invention is not limited in its application to the details of construction and the arrangement of components set forth in the following description or illustrated in the drawings. The present invention is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

The compounds disclosed herein may also contain unnatural proportions of atomic isotopes of one or more of the atoms that constitute such compounds. For example, the compounds may be radiolabeled with radioactive isotopes, such as for example tritium (H), iodine-125 (′I) or carbon-14 (″C). All isotopic variations of the compounds of the present invention, whether radioactive or not, are intended to be encompassed within the scope of the present invention.

Selected compounds having a formal electronic charge may be shown without an appropriate biologically compatible counterion. Such a counterion serves to balance the positive or negative charge present on the compound. As used herein, the substance that is biologically compatible is non-toxic as used, and does not have substantially deleterious effects on biomolecules.

Examples of negatively charged counterions include, among others, chloride, bromide, iodide, Sulfate, alkanesulfonate, arylsulfonate, phosphate, perchlorate, tetrafluoroborate, tetraarylboride, nitrate and anions of aromatic or aliphatic carboxylic acids. Preferred counterions may include chloride, iodide, perchlorate and various Sulfonates. Examples of positively charged counterions include, among others, alkali metal, or alkaline earth metal ions, ammonium, or alkylammonium ions.

All documents cited in the present specification are hereby incorporated by reference in their totality. In particular, the teachings of all documents herein specifically referred to are incorporated by reference.

Exemplary embodiments of the present invention are described with reference to the accompanying figures.

In the drawings, like reference numbers, generally indicate identical, functionally similar, and/or structurally similar elements. The drawing in which an element first appears is indicated by the leftmost digit(s) in the corresponding reference number.

The invention is further illustrated by the following examples which in no way should be construed as being further limiting. One skilled in the art will readily appreciate that the specific methods and results described are merely illustrative. Structures of the intermediates as well as the final compounds were confirmed by nuclear magnetic resonance spectra for proton (H NMR) and LCMS.

### Definitions

Hydrogen means ‘H’ atoms and its radio isotopes deuterium, tritium as per the requirement.

Alkyl means a group containing one hydrogen less than the corresponding saturated hydrocarbon that is straight chain, branched, having substituents, or forming part of another molecule.

Halogen means “F”, “Cl”, “Br”, “I” atoms as substituents directly or attached to other moieties.

Haloalkyl refers to an alkyl group containing halogen atoms.

Aryl” means a phenyl, naphthyl, biphenyl or indenyl group.

Hydroxyl means presence of one or more (—OH—) groups as substituents.

“Heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens).

“Cycloalkyl” means any mono- or bi-cyclic non-aromatic carbocyclic group containing from 3 to 10 ring members, which may include fused, bridged or spiro ring systems.

“Heterocycloalkyl” means any mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. It being possible for the aryl, heteroaryl, cycloalkyl and heterocycloalkyl groups so defined and the alkyl, alkenyl, alkynyl, alkoxy, to be substituted by from 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆) polyhaloalkyl; trifluoromethoxy; (C₁-C₆) alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and addition salts thereof with a pharmaceutically acceptable acid or base.

Substituent groups are specified by their conventional chemical formulae, written from left to right, they equally encompass the chemically identical Substituents, which would result from writing the structure from right to left, e.g., —CH2O— is intended to also recite —OCH2-.

The term “acyl or “alkanoyl by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical’ is the group derived from a carboxylic acid by removing the OH moiety there from.

The term “alkyl.” by itself or as part of another substituent means, a straight or branched chain, or cyclic hydrocarbon radical, or combination thereof, which may be fully saturated, mono- or polyunsaturated and can include divalent (“alkylene’) and multivalent radicals, having the number of carbon atoms designated.

Saturated hydrocarbon radicals include, but are not limited to, groups such as methyl, ethyl, n-propyl, isopropyl. n-butyl, t-butyl, isobutyl, sec-butyl, cyclohexyl, (cyclohexyl)methyl, cyclopropylmethyl.

Homologues and isomers of for example, n-pentyl, n-hexyl, n-heptyl, n-octyl, and the like.

An unsaturated alkyl group is one having one or more double bonds(alkenyl) or triple bonds (alkynyl). Examples of unsaturated alkyl groups include, but are not limited to, vinyl, 2-propenyl, crotyl, 2-isopentenyl, 2-(butadienyl), 2.4-pentadienyl, 3-(1,4-pentadienyl), ethynyl, 1- and 3-propynyl, 3-butynyl, and the higher homologues and isomers.

The term “alkyl, unless otherwise noted, is also meant to include those derivatives of alkyl defined in more detail below, such as “heteroalkyl. Alkyl groups that are limited to hydrocarbon groups are termed “homoalkyl.

The terms “cycloalkyl and “heterocyclylalkyl, by themselves or in combination with other terms, represent, unless otherwise stated, cyclic versions of “alkyl and “heteroalkyl, respectively. Additionally, for heterocycloalkyl, a heteroatom can occupy the position at which the heterocycle is attached to the remainder of the molecule.

The terms “alkoxy.” “alkylamino” and “alkylthio’ (or thioalkoxy) are used in their conventional sense, and refer to those alkyl groups attached to the remainder of the molecule via an oxygen atom, an amino group, or a sulfur atom, respectively.

The term “amino” or “amine group’ refers to the group —NR′R″ (or NRRR″) where R, R and R″ are independently selected from the group consisting of hydrogen, alkyl, substituted alkyl, aryl, substituted aryl, arylalkyl, substituted aryl alkyl, heteroaryl, and substituted heteroaryl, acyl constituting amino, alkylamino, arylamino and heteroarylamino, acylamino groups. In addition, the terms “amine’ and “amino” can include protonated and quaternized versions of nitrogen, comprising the group —NRRR″ and its biologically compatible anionic counterions.

Substituted —SO₂ refers to —SO₂-alkyl or aryl or heteroaryl, SR, —SOR, —SO₂R, and R in each of the above groups can be hydrogen, substituted or unsubstituted alkyl, substituted or, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted heteroaryl, substituted heterocyclylalkyl ring, substituted or unsubstituted heteroarylalkyl, or substituted or unsubstituted heterocyclic ring, or any two of R groups may be joined to form a substituted or unsubstituted saturated or unsaturated 3-10 membered ring, which may optionally include hetero atoms which may be the same or different and are selected from O, NR (wherein R is hydrogen or substituted or unsubstituted alkyl or S).

Acyl group is derived by the removal of one or more hydroxyl groups from an oxoacid, including inorganic acids The general formula is RCO—, where R represents an alkyl, substituted group that is linked to the carbon atom of the group by a single bond and can also include sulfonic acids, phosphonic acids.

Nucleic acids mean deoxyribonucleic acid (DNA), and ribonucleic acid (RNA).

RT (Or rt) means room temperature.

RT-PCR means Reverse transcription polymerase chain reaction.

Fluorescent means capable of fluorescence when excited at an appropriate wavelength of light.

Nucleic acid staining agents find extensive application in detection, imagery, monitoring, cancer treatment, detection of abnormalities, forensics, assisted reproduction methods etc. Conventionally the nucleic acid staining agents should be amenable to gel electrophoresis to facilitate the staining and subsequent detection process. These staining agents are commonly flurophores that exhibit fluorescence in UV or visible light due to energy transfer. The energy transfer occurs due to electron transfer between the reactive moieties present on the nucleic acid staining agent molecules and the nucleic acid base pairs while binding, which is further detected by suitable techniques such as electrofluorimetry.

The staining agent does not interfere with the mobility of the nucleic acid as it does not intercalate in which case it can be employed in the pre-loading technique, or it can be used as a post-loading agent. The staining agent should possess at least 2 or more groups that can facilitate binding with the nucleic acid base pairs (Alicia M. Haines et al, properties of nucleic acid staining dyes used in gel electrophoresis, Electrophoresis 36(6), 2014) and mostly polycyclic, especially compounds with 2-6 cyclic groups are preferred in this regard. Heterocyclic compounds in fusion with aromatic or alicyclic (cycloalkane) rings are considered to be suitable candidates to act as fluorescent indicators owing to the presence of 1 to 4 atoms selected from O, N, S which are responsible for energy transfer in view of their electron richness.

The nucleic acid staining agents which are in use at present have a few shortcomings such as toxicity, mutagenicity, either double or single strand binding, lack of sensitivity, non-applicability to nucleic acid detection other than the nucleus, false-positivity/negativity, expensive nature, problem of non-conforming to gel-electrophoresis, problems of toxic waste disposal etc. In order to overcome these shortcomings and also to come out with nucleic acid staining agents that can be used to detect all forms of nucleic acids, along with proteins, cell-organelles in a non-toxic and cost-effective manner, this invention is taken up.

The present invention discloses the methods of preparation of fused tetracyclic, heterocyclic compounds of formula (I), its salts, derivatives, tautomers, polymorphs, stereoisomers, solvates, and its applications in the detection of nucleic acids. The invention can be realized according to the detailed synthetic routes given hereunder and non-limiting within the scope of this invention and is useful as nucleic acid staining agents. The compounds of formula (I) are prepared following independent general synthetic routes as outlined in the Schemes.

According to a first aspect, Fluorescent nucleic acid staining agents comprising fused tetracyclic, heterocyclic compounds of formula (I), their tautomers, polymorphs, stereoisomers, solvates are provided.

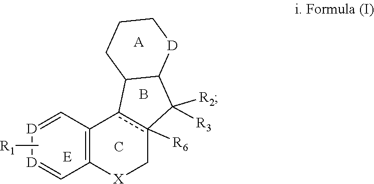

A is polycyclic heterocyclic ring which is unsaturated or partially unsaturated, optionally having up to two heteroatoms independently selected from O, N or S;
Ring A can be optionally substituted by the atoms or groups comprising hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
D is selected from CR⁴ or N;
X is selected from CR⁴, O, NR⁵ or S;
ring C is saturated or partially unsaturated, when it is saturated R6 can be H or OH whereas in case when ring C is partially unsaturated R6 is absent;
R¹ and R⁴ are selected independently from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
R¹ and R⁴ can cyclize to form a 4-7 membered ring which can be optionally substituted by hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
R² and R³ are independently selected from hydrogen, alkyl, cycloalkyl alkenyl, alkynyl, alkoxy, acyl, acylamino, optionally R² and R³ can combine to form a 3 to 7 membered ring; a. R⁵ is selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl.

In an embodiment, the compound of formula (I) is 9,10-trimethoxy-5-(p-tolylsulfonyl)-7,11b-dihydro-6H-indeno[2,1-c]quinolin-6a-ol.

In another embodiment, the compound of formula (I) is 5-(p-tolylsulfonyl)-7,11b-dihydro-6H-indeno[2,1-c]quinoline-3,6a,9,10-tetrol.

In yet another embodiment, the compound of formula (I) is (3Z)-3-[(3,4-dimethoxyphenyl)methylene]-7-methoxy-thiochroman-4-one.

In yet another embodiment, the compound of formula (I) is 3,9,10-trimethoxy-6,7-dihydroindeno[2,1-c]thiochromene (Isomer-1).

In yet another embodiment, the compound of formula (I) is 3,9,10-trimethoxy-6,7-dihydroindeno[2,1-c]thiochromene:—(Isomer-2).

In yet another embodiment, the compound of formula (I) is 3,9,10-trimethoxy-6,7-dihydroindeno[2,1-c]thiochromene.

In yet another embodiment, the compound of formula (I) is 2-bromo-3,9,10-trimethoxy-7,11b-dihydro-6H-indeno[2,1-c]chromen-6a-ol.

In yet another embodiment, the compound of formula (I) is 2-bromo-7,11b-dihydro-6H-indeno[2,1-c]chromene-3,6a,9,10-tetrol.

In yet another embodiment, the compound of formula (I) is 3,9,10-trimethoxy-2-phenyl-7,11b-dihydro-6H-indeno[2,1-c]chromen-6a-ol.

In yet another embodiment, the compound of formula (I) is 2-phenyl-7,11b-dihydro-6H-indeno[2,1-c]chromene-3,6a,9,10-tetrol.

In yet another embodiment, the compound of formula (I) is 2-(4-fluorophenyl)-3,9,10-trimethoxy-7,11b-dihydro-6H-indeno[2,1-c]chromen-6a-ol.

In yet another embodiment, the compound of formula (I) is 2-(4-fluorophenyl)-7,11b-dihydro-6H-indeno[2,1-c]chromene-3,6a,9,10-tetrol.

In yet another embodiment, the compound of formula (I) is 2-bromo-3,9,10-trimethoxy-5-(p-tolylsulfonyl)-7,11b-dihydro-6H-indeno[2,1-c]quinolin-6a-ol.

In yet another embodiment, the compound of formula (I) is 3,9,10-trimethoxy-2-phenyl-5-(p-tolylsulfonyl)-7,11b-dihydro-6H-indeno[2,1-c]quinolin-6a-ol.

In yet another embodiment, the compound of formula (I) is 3,9,10-trimethoxy-2-phenyl-5-(p-tolylsulfonyl)-7,11b-dihydro-6H-indeno[2,1-c]quinolin-6a-ol.

In yet another embodiment, the compound of formula (I) is 2-(4-fluorophenyl)-3,9,10-trimethoxy-5-(p-tolylsulfonyl)-7,11b-dihydro-6H-indeno[2,1-c]quinolin-6a-ol.

In yet another embodiment, the compound of formula (I) is 2-(4-fluorophenyl)-5-(p-tolylsulfonyl)-7,11b-dihydro-6H-indeno[2,1-c]quinoline-3,6a,9,10-tetrol.

In yet another embodiment, the compound of formula (I) is 3-(3,4-dimethoxybenzyl)-7-methoxy-3,4-dihydro-2H-chromene-3,4-diol.

In yet another embodiment, the compound of formula (I) comprises prophetic molecules 1-24 as given in Table 1 with the following structures:

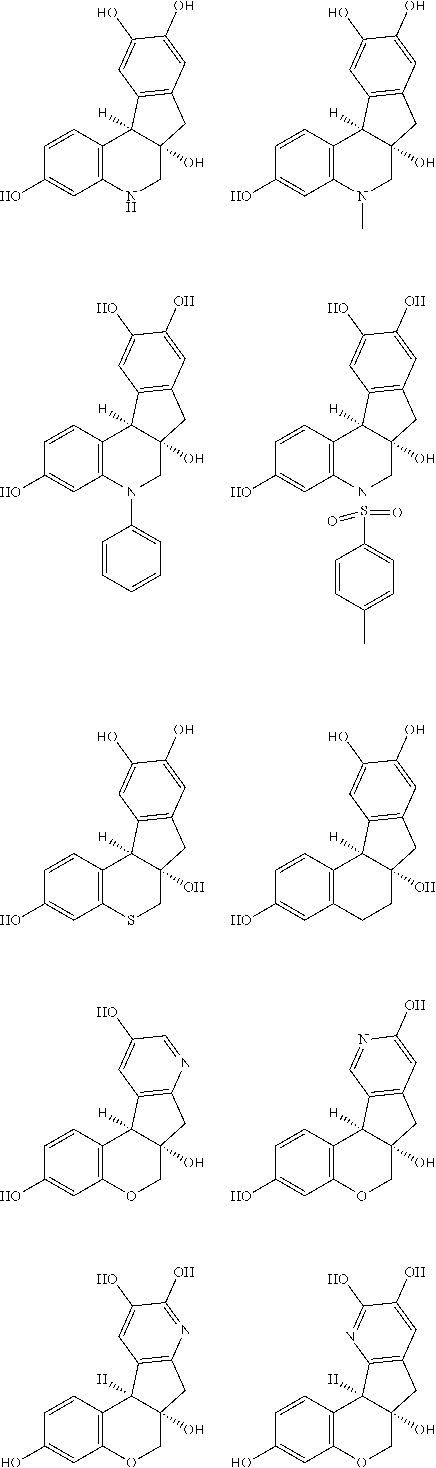

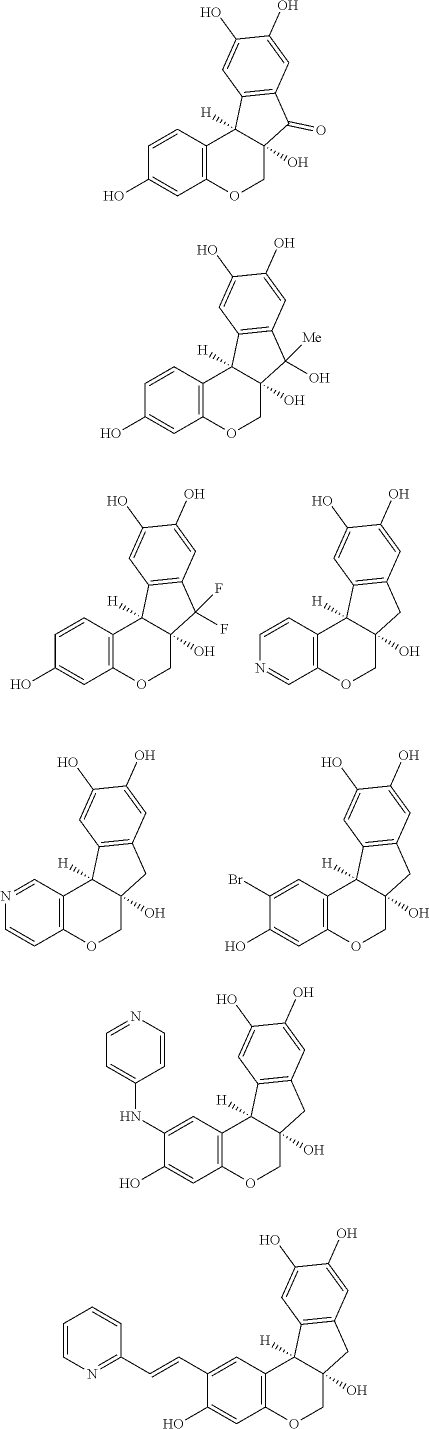

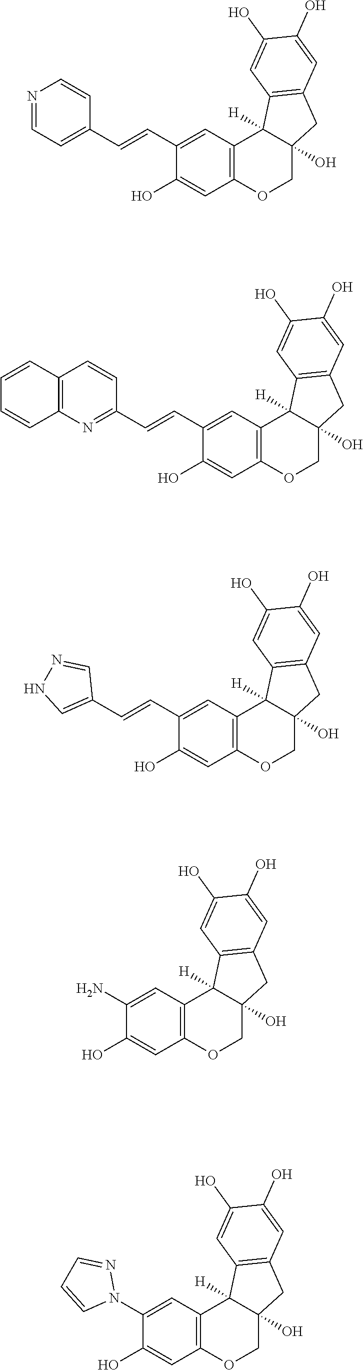 [SMILES: Oc1cc2c(cc1O)[C@@H]1c3cc(/C=C/c4ccncc4)c(O)cc3OC[C@]1(O)C2 ; Oc1cc2c(cc1O)[C@@H]1c3cc(/C=C/c4ccc5ccccc5n4)c(O)cc3OC[C@]1(O)C2 ; Oc1cc2c(cc1O)[C@@H]1c3cc(/C=C/c4cn[nH]c4)c(O)cc3OC[C@]1(O)C2 ; Nc1cc2c(cc1O)OC[C@]1(O)Cc3cc(O)c(O)cc3[C@H]21 ; Oc1cc2c(cc1O)[C@@H]1c3cc(-n4cccn4)c(O)cc3OC[C@]1(O)C2]

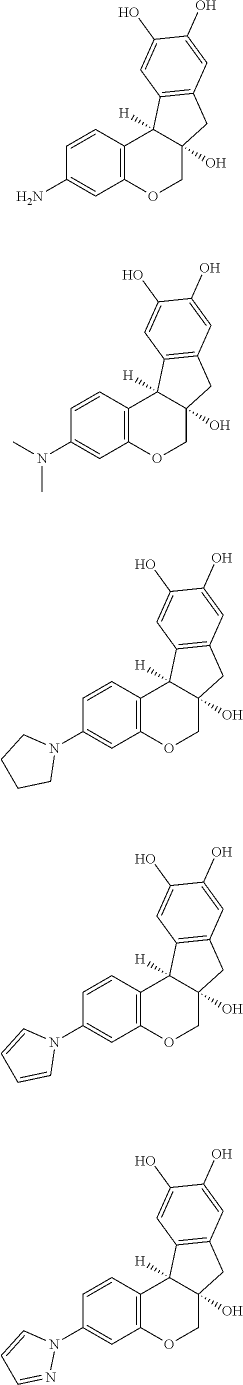 [SMILES: Nc1ccc2c(c1)OC[C@]1(O)Cc3cc(O)c(O)cc3[C@H]21 ; CN(C)c1ccc2c(c1)OC[C@]1(O)Cc3cc(O)c(O)cc3[C@H]21 ; Oc1cc2c(cc1O)[C@@H]1c3ccc(N4CCCC4)cc3OC[C@]1(O)C2 ; Oc1cc2c(cc1O)[C@@H]1c3ccc(-n4cccc4)cc3OC[C@]1(O)C2 ; Oc1cc2c(cc1O)[C@@H]1c3ccc(-n4cccn4)cc3OC[C@]1(O)C2]

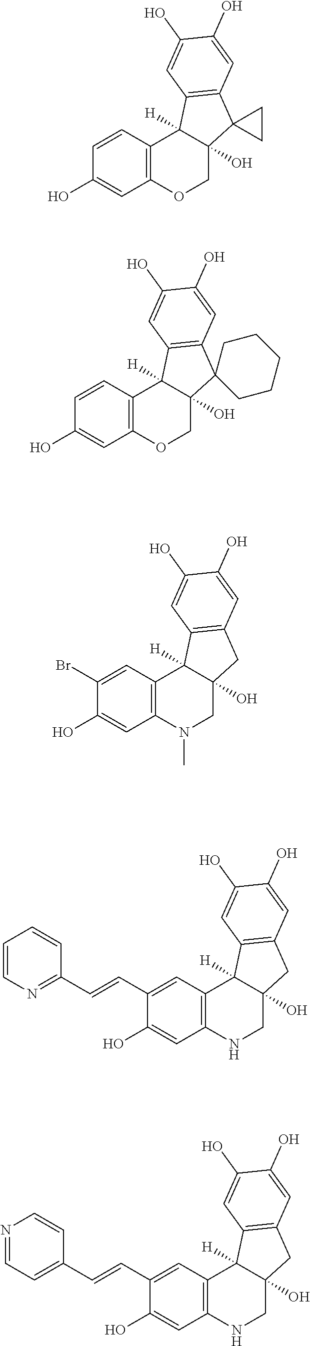 [SMILES: Oc1ccc2c(c1)OC[C@@]1(O)[C@@H]2c2cc(O)c(O)cc2C12CC2 ; Oc1ccc2c(c1)OC[C@@]1(O)[C@@H]2c2cc(O)c(O)cc2C12CCCCC2 ; CN1C[C@]2(O)Cc3cc(O)c(O)cc3[C@@H]2c2cc(Br)c(O)cc21 ; Oc1cc2c(cc1O)[C@@H]1c3cc(/C=C/c4ccccn4)c(O)cc3NC[C@]1(O)C2 ; Oc1cc2c(cc1O)[C@@H]1c3cc(/C=C/c4ccncc4)c(O)cc3NC[C@]1(O)C2]

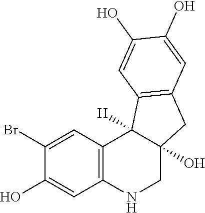 [SMILES: Oc1cc2c(cc1O)[C@@H]1c3cc(Br)c(O)cc3NC[C@]1(O)C2]

In yet another embodiment, the fused tetracyclic, heterocyclic fluorescent nucleic acid staining agents with the structure as given in formula (I), where R¹ and R⁴ are additionally selected from a group comprising arylalkyl, heteroaryl, heteroarylalkyl, cycloalkyl, cycloalkylalkyl, heterocyclyl or heterocyclylalkyl, mono- or bi-cyclic non-aromatic carbocyclic group composed of 3 to 10 ring members, and containing one to 3 hetero atoms selected from oxygen, sulphur, SO, SO₂ and nitrogen and further optionally,
(a) it being understood that bicyclic group may be fused or spiro type,
(b) it being possible for the aryl, heteroaryl, cycloalkyl and heterocycloalkyl groups so defined and the alkyl, alkenyl, alkynyl, alkoxy to be substituted by 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆)polyhaloalkyl; trifluoromethoxy; (C₁-C₆)alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and additionally salts thereof with a pharmaceutically acceptable acid or base.

In yet another embodiment, the fused tetracyclic, heterocyclic fluorescent nucleic acid staining agents with the structure as given in formula (I), where R¹ and R⁴ cyclize to form a 4-7 membered ring which can be additionally substituted by mono- or bi-cyclic non-aromatic carbocyclic group, composed of 3 to 10 ring members, and containing one to 3 hetero atoms selected from oxygen, sulphur, SO, SO₂ and nitrogen and additionally,
(a) it being understood that bicyclic group may be fused or spiro type,
(b) it being possible for the aryl, heteroaryl, cycloalkyl and heterocycloalkyl groups so defined and the alkyl, alkenyl, alkynyl, alkoxy, to be substituted by from 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆)polyhaloalkyl; trifluoromethoxy; (C₁-C₆)alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and addition salts thereof with a pharmaceutically acceptable acid or base.

In yet another embodiment, the fused tetracyclic, heterocyclic fluorescent nucleic acid staining agents with the structure as given in formula (I), where R² and R³ are independently selected from hydrogen, alkyl, cycloalkyl alkenyl, alkynyl, alkoxy, acyl, acylamino, amino or amine group consisting of hydrogen, alkyl, substituted alkyl, aryl, substituted aryl, arylalkyl, substituted aryl alkyl, heteroaryl, and substituted heteroaryl, acyl constituting amino, alkylamino, arylamino, heteroarylamino, acylamino groups, including protonated and quaternized nitrogen comprising the group —NRRR″ and its biologically compatible anionic counterions.

In yet another embodiment, the fused tetracyclic, heterocyclic fluorescent nucleic acid staining agents with the structure as given in formula (I), where R⁵ is optionally selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl or optionally substituted —SO₂ moiety;
wherein, optionally substituted —SO₂ comprising —SO₂-alkyl or aryl or heteroaryl, SR, —SOR, —SO₂R, and R in each of the above groups selected independently from hydrogen, substituted or unsubstituted alkyl, substituted or, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted heteroaryl, substituted heterocyclylalkyl ring, substituted or unsubstituted heteroarylalkyl, or substituted or unsubstituted heterocyclic ring, or any two of R groups joined to form a substituted or unsubstituted saturated or unsaturated 3-10 membered ring, which may optionally include hetero atoms which may be the same or different and are selected from O, NR (wherein R is hydrogen or substituted or unsubstituted alkyl or S).

In yet another embodiment, the fused tetracyclic, heterocyclic fluorescent nucleic acid staining agents with the structure as given in formula (I) are nucleic acid staining agents to detect nucleic acids from nucleus and non-nucleus cell organelles, from biological samples, scenes of crime, imagery.

In yet another embodiment, the fused tetracyclic, heterocyclic fluorescent nucleic acid staining agents with the structure as given in formula (I) are fluorescent nucleic acid staining agents to detect proteins, DNA, RNA from cell-lines, cell organelles, mitochondria, fragments, body fluids, tissues, biopsies, swabs, plants, animals, humans, and yeasts.

In yet another embodiment, the fused tetracyclic, heterocyclic fluorescent nucleic acid staining agents with the structure as given in formula (I) are highly specific, and sensitive fluorescent nucleic acid staining agents, that can detect single-strand, double-strand DNA, RNA, Proteins.

In yet another embodiment, the fused tetracyclic, heterocyclic fluorescent nucleic acid staining agents with the structure as given in formula (I) are used for mass-screening, rapid-screening, monitoring of analytes in case of diseases such as cancer, targeted therapy, fossil-analysis, forensics, and biological profiling.

In yet another embodiment, tautomers, stereoisomers, solvates, polymorphs of the structure as given in formula (I) are formed using generally acceptable inorganic and organic acids.

In yet another embodiment, the compounds of formula (I) finding application as nucleic acid staining agents are non-intercalating, non-mutagenic, and non-toxic.

In yet another embodiment, the compound of formula (I) is produced according to any one of the schemes 1-6.

In yet another embodiment, the fluorescent fused tetracyclic, heterocyclic nucleic acid staining compounds of formula (I) possesses sulphur moieties to facilitate binding to nucleic acids and proteins.

According to another aspect a method of detection of nucleic acids by compounds of formula (I) using UV excitation or other spectrofluorimetric methods of analysis is provided.

In an embodiment, the method of detection of nucleic acids by fluorescent nucleic acid staining by compounds of formula (I) consisted of a detection apparatus comprising gel documentation system, UV transilluminator or spectrofluorimeter.

According to the second aspect, a diagnostic kit comprising compounds of formula (I) as fluorescent nucleic acid staining agents, fluorescent lamp, UV-spectrofluorometer and gel documentation is provided. The gel is agarose gel.

According to a third aspect, a method of synthesizing the compounds of formula (I) is provided. The method comprising three steps A, B, and C. Step A comprises;
a. synthesis of the intermediate IV from aryl-XH with the structure as given,
b. by Michael addition of aryl-XH and alkyl acrylate/acrylonitrile using bases selected from a group comprising NaH, KOtBu, NaOtBu, K2CO3,
   or by alkylation of the compound having the structure of (I) using strong bases to give compound II;
   iii) compound II undergoing ester hydrolysis in presence of bases comprising LiOH, NaOH in the solvent media comprising THF, MeOH, water or mixture thereof.
   iv) Lewis acid mediated cyclization of compound II leading to compound IV wherein Lewis acids comprising poly phosphoric acid (PPA) or trifluoroacetic acid (TFA) or methanesulfonic acid (MSA), pTSA either in catalytic or stoichiometric amounts are employed for mediating cyclization of compound II to yield compound III and cyclization of II in presence of Lewis acids yielding the intermediate having structure as given in IV;
   Step B comprises;
   ia) synthesis of compound VI via aldol condensation of corresponding aldehydes and compound IV in the presence of bases comprising NaOH, KOH or acids such as H₂SO₄, triflic acid, acetic acid, HCl or
   ib) by enamine chemistry using a group comprising piperidine/or pyrrolidine/or any secondary aliphatic amine,
   ii) epoxidation of exocyclic double bonds of compound VI in presence of oxidizing agents selected from a group comprising H₂O₂, Oxone, TBHP, mCPBA, etc. in the presence/or absence of a base to obtain compound VII,
   iii) cyclization of VII to tetracyclic compound IX controlled by acids selected from a group comprising perchloric acid, acetic acid, hydrochloric acid, sulphuric acid, triflic acid, trifluoroacetic acid independently or a mixture thereof in the presence/or absence of solvent such as methanol, ethanol, THF, dioxane, toluene, xylene, etc.
   Step C comprises;
   synthetic routes for the derivatization of compound XI for obtaining compounds of formula (I) by coupling reactions using catalysts such as palladium in the presence of suitable phosphine ligands of the commercially available palladium phosphine complex as given under wherein;
   Obtaining compound XII from compound XI using appropriate amine as a coupling partner with compound XI under conditions known in the literature for Buchwald-Hartwig amination which upon deprotection resulting in compound XV,
   using compound XI and appropriate catalysts such as boronic acid or boronates under Suzuki coupling conditions for obtaining compound XIII which upon deprotection yielding compound XV,
   coupling of compound XI under Heck coupling conditions with appropriate olefins to obtain alkene substituted derivative XIV, which upon further deprotection resulting in compound XVII.

According to an embodiment, the method yields compounds of formula(I) with suitable moieties in their structure on derivatization in Step C of the method using suitable derivatizing agents, yield fused tetracyclic, heterocyclic fluorescent nucleic acid staining agents to detect nucleic acids from analytes.

According to another embodiment, the method includes exemplary synthesis of 3-(3,4-dimethoxybenzyl)-7-methoxy-3,4-dihydro-2H-chromene-3,4-diol from Resorcinol and chloro-propionic acid.

According to another embodiment, the compounds of formula (I) as claimed comprising molecules according to above mentioned with the structures given.

### 1. General Schemes

### General Synthetic Route for the Preparation of Compounds of Formula (I)

The general synthetic route for the preparation of compounds of formula(I) involved 3 steps. The first step A involves the synthesis of the intermediate IV with the structure as given in the step A Intermediate IV can be synthesized by 2 different routes employing Michael addition of aryl-XH and alkyl acrylate or alkylation of the compound having the structure of I using strong bases to give compound II which undergoes hydrolysis to yield compound III and cyclization of II in presence of acid will yield the intermediate having structure given in IV. Compound IV undergoes aldol condensation in presence of strong bases with corresponding aldehyde yields compound VI as given in step B, which undergoes exocyclic epoxidation to yield compound with the structure of VII, which further cyclizes to yield compounds of formula (I) in presence of oxidizing agents and inorganic acids in presence or absence of solvents. Step C involves derivatization of compounds of formula (I) with suitable coupling reagents known in the literature. The following schematic gives a detailed procedure of the different steps involved in synthesizing the compounds of formula (I).

### A) Synthesis of Intermediate IV:

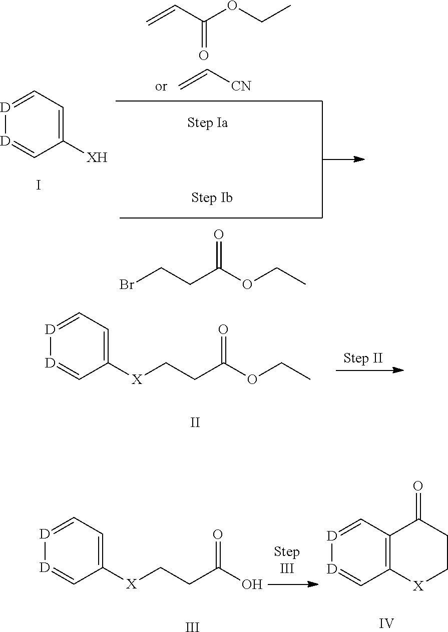

As shown in scheme A, compound II can be synthesized via 1,4 conjugate addition (Michael addition) reaction of aryl-XH and alkyl acrylate or acrylonitrile using base such as NaH, KOtBu, NaOtBu, K2CO3. Compound II can also be alternatively synthesized via alkylation using base as mentioned above. Compound III can be synthesized via ester hydrolysis using LiOH or NaOH in the solvent media such as THF, MeOH, Water or mixture thereof. Acid mediated cyclization of compound II can lead to compound IV. Any lewis acid may be used for such cyclization e. g. poly phosphoric acid (PPA) or trifluoroacetic acid (TFA) or methanesufonic acid (MSA), pTSA either in catalytic or stoichiometric amount.

### B) Synthesis of Compounds of Formula (I):

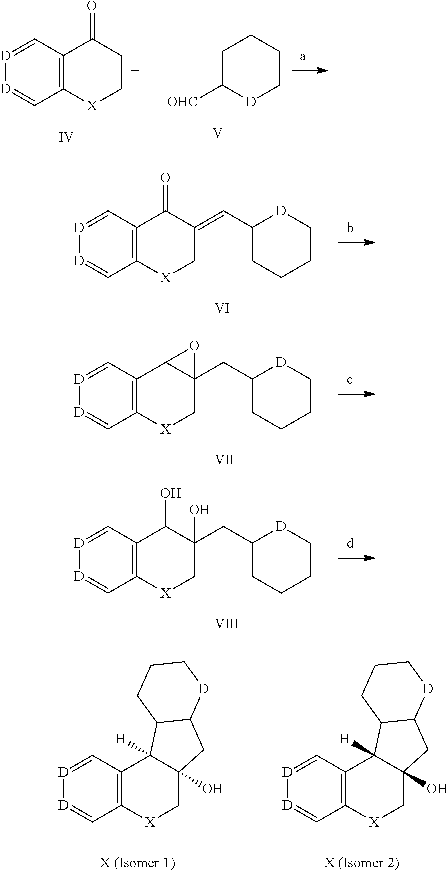

As shown in scheme B, compound VI can be synthesized via aldol condensation of corresponding aldehyde and compound IV in the presence of bases such as NaOH, KOH or acids such as H₂SO₄, triflic acid, acetic acid, HCl or by enamine chemistry using piperidine or pyrrolidine or any secondary aliphatic amine. Epoxidation of exocyclic double bonds of compound VI can be carried out using any oxidizing agent such as H₂O₂, Oxone, TBHP, mCPBA, etc in the presence or absence of base to obtain compound VII. Further it can be cyclized to tetracyclic compound IX using perchloric acid, acetic acid, hydrochloric acid, sulphuric acid, triflic acid, trifluoroacetic acid independently or a mixture thereof in the presence or absence of solvent such as methanol, ethanol, THF, dioxane, toluene, xylene, etc.

### C) Synthesis of Compounds of Formula (I):

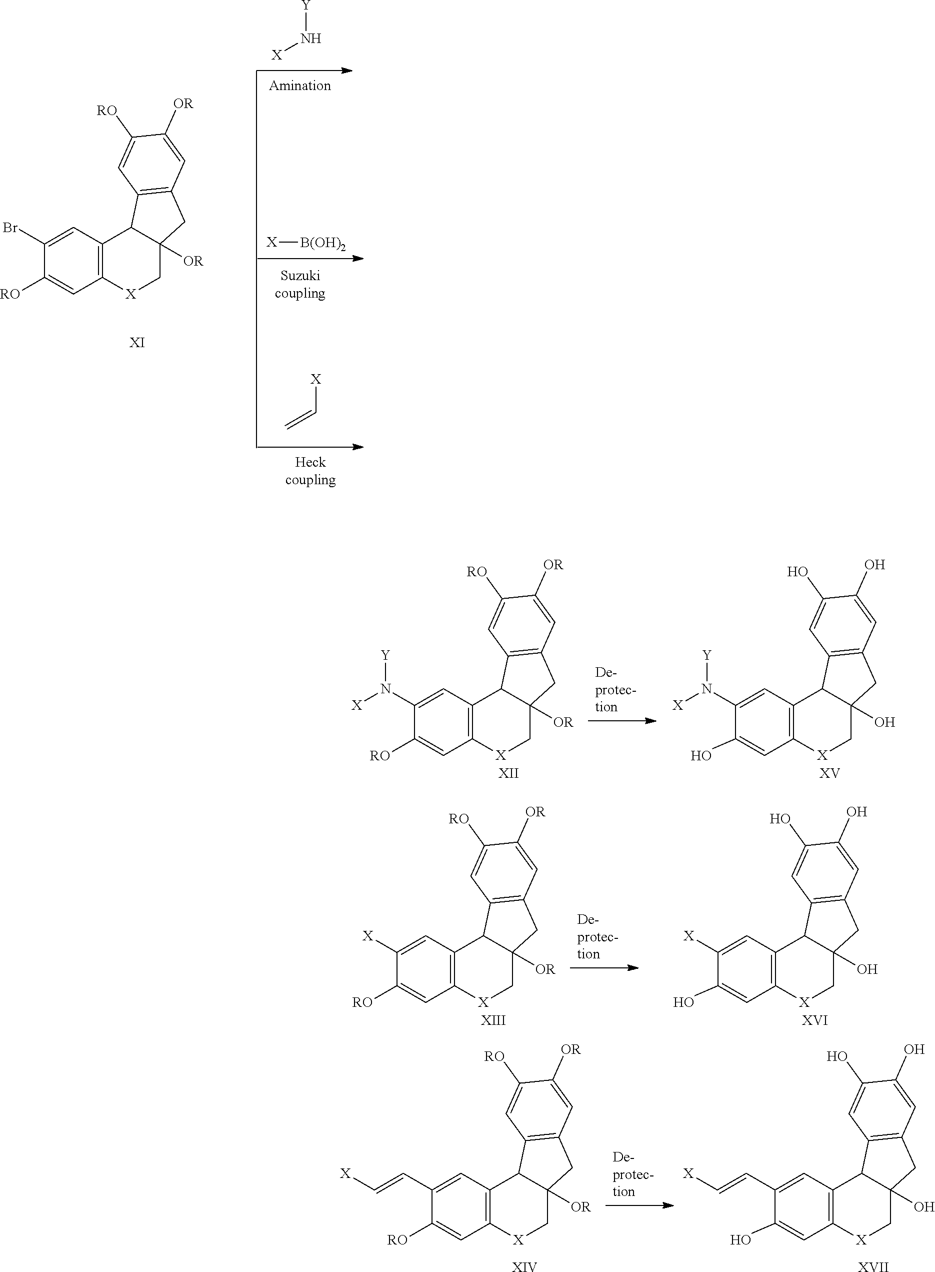

Scheme C represents the general synthetic route for the derivatization of comp XI. Using appropriate amine as a coupling partner with comp XI and conditions known in the literature for Buchwald-Hartwig amination can give comp XII which upon deprotection can lead to comp XV. Similarly using comp XI and appropriate boronic acid or boronates under Suzuki coupling conditions can lead to comp XIII which upon deprotection can give comp XVI. Under Heck coupling conditions comp XI can be coupled with appropriate olefin to obtain alkene substituted derivative XIV, which upon further deprotection can provide comp XVII. For the above-mentioned coupling reactions, the catalyst such as palladium in the presence of a suitable phosphine ligand of the commercially available palladium phosphine complex can be used.

By using the suitable starting compounds with specific moieties, the fused tetracyclic, heterocyclic compounds thus obtained from the general synthetic route consisting of steps A, B and further derivatizing the obtained compounds one can synthesize compounds of formula (I) as given by this invention for their application as nucleic acid staining agents. The fused tetracyclic, heterocyclic nucleic acid staining agents thus synthesized will have a general formula as given in the structure below wherein,

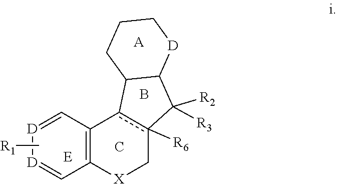

### 1. Formula (I)

A is polycyclic heterocyclic ring which is unsaturated or partially unsaturated optionally having up to two heteroatoms independently selected from O, N or S; Ring A can be optionally substituted by the atoms or group selected from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl
D is selected from CR⁴ or N
X is selected from CR⁴, O, NR⁵ or S
ring C is saturated or partially unsaturated; when it is saturated, R6 can be H or OH whereas in case ring C is partially unsaturated R6 is absent.
R¹ and R⁴ are selected from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. It being possible for the aryl, heteroaryl, cycloalkyl and heterocycloalkyl groups so defined and the alkyl, alkenyl, alkynyl, alkoxy, to be substituted by from 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆) polyhaloalkyl; trifluoromethoxy; (C₁-C₆) alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and addition salts thereof with a pharmaceutically acceptable acid or base.

R¹ and R⁴ can cyclize to form a 4-7 membered ring which can be optionally substituted by hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl. mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. It being possible for the aryl, heteroaryl, cycloalkyl and heterocycloalkyl groups so defined and the alkyl, alkenyl, alkynyl, alkoxy, to be substituted by from 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆)polyhaloalkyl; trifluoromethoxy; (C₁-C₆)alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and addition salts thereof with a pharmaceutically acceptable acid or base.

R² and R³ are independently selected from hydrogen, alkyl, cycloalkyl alkenyl, alkynyl, alkoxy, acyl, acylamino, amino or amine group consisting of hydrogen, alkyl, substituted alkyl, aryl, substituted aryl, arylalkyl, substituted aryl alkyl, heteroaryl, and substituted heteroaryl, acyl constituting amino, alkylamino, arylamino and heteroarylamino, acylamino groups. In addition, the terms “amine’ and “amino” can include protonated and quaternized versions of nitrogen, comprising the group —NRRR″ and its biologically compatible anionic counterions optionally R² and R³ can combine to form a 3 to 7 membered ring.

R⁵ is selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl. Optionally Substituted —SO₂ refers to —SO₂-alkyl or aryl or heteroaryl, SR, —SOR, —SO₂R, and R in each of the above groups can be hydrogen, substituted or unsubstituted alkyl, substituted or, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted heteroaryl, substituted heterocyclylalkyl ring, substituted or unsubstituted heteroarylalkyl, or substituted or unsubstituted heterocyclic ring, or any two of R groups may be joined to form a substituted or unsubstituted saturated or unsaturated 3-10 membered ring, which may optionally include hetero atoms which may be the same or different and are selected from O, NR (wherein R is hydrogen or substituted or unsubstituted alkyl or S).

In an exemplary aspect of the present invention of compounds of formula (I), the application of the compounds as nucleic acid staining agents from nucleus and non-nucleus cell organelles from biological samples, scenes of crime, imagery.

In another exemplary aspect the invention of compounds of formula (I) are its tautomers, stereoisomers, solvates, polymorphs, formed using generally acceptable inorganic and organic acids such hydrochloric acid, sulfonic acids etc.

In one exemplary aspect of the invention of compounds of formula (I), the invention discloses the preparation and application of prophetic molecules numbered 1-34 along with their structures as given in table 1 and isomers 1 and 2 and also Brazilin

Yet another important aspect of the invention of compounds of formula (I) the compounds finding application as nucleic acid staining agents are non-intercalating which shows their non-mutagenicity and non-toxic nature.

The present invention specifically discloses the synthesis and use as nucleic acid staining agents of the following compounds according to the detailed procedures given in the disclosure:
,9,10-trimethoxy-5-(p-tolylsulfonyl)-7,11b-dihydro-6H-indeno[2,1-c]quinolin-6a-ol (11):
5-(p-tolylsulfonyl)-7,11b-dihydro-6H-indeno[2,1-c]quinoline-3,6a,9,10-tetrol (12):
(3Z)-3-[(3,4-dimethoxyphenyl)methylene]-7-methoxy-thiochroman-4-one (17)
3,9,10-trimethoxy-6,7-dihydroindeno[2,1-c]thiochromene (20) (Isomer-1)
3,9,10-trimethoxy-6,7-dihydroindeno[2,1-c]thiochromene:—(Isomer-2)
3,9,10-trimethoxy-6,7-dihydroindeno[2,1-c]thiochromene (21)
2-bromo-3,9,10-trimethoxy-7,11b-dihydro-6H-indeno[2,1-c]chromen-6a-ol (32):
-bromo-7,11b-dihydro-6H-indeno[2,1-c]chromene-3,6a,9,10-tetrol (33):
3,9,10-trimethoxy-2-phenyl-7,11b-dihydro-6H-indeno[2,1-c]chromen-6a-ol (34)
2-phenyl-7,11b-dihydro-6H-indeno[2,1-c]chromene-3,6a,9,10-tetrol (35)
2-(4-fluorophenyl)-3,9,10-trimethoxy-7,11b-dihydro-6H-indeno[2,1-c]chromen-6a-ol (36)
2-(4-fluorophenyl)-7,11b-dihydro-6H-indeno[2,1-c]chromene-3,6a,9,10-tetrol (37)
2-bromo-3,9,10-trimethoxy-5-(p-tolylsulfonyl)-7,11b-dihydro-6H-indeno[2,1-c]quinolin-6a-ol (42)
3,9,10-trimethoxy-2-phenyl-5-(p-tolylsulfonyl)-7,11b-dihydro-6H-indeno[2,1-c]quinolin-6a-ol (44)
3,9,10-trimethoxy-2-phenyl-5-(p-tolylsulfonyl)-7,11b-dihydro-6H-indeno[2,1-c]quinolin-6a-ol (45)
2-(4-fluorophenyl)-3,9,10-trimethoxy-5-(p-tolylsulfonyl)-7,11b-dihydro-6H-indeno[2,1-c]quinolin-6a-ol (46)
2-(4-fluorophenyl)-5-(p-tolylsulfonyl)-7,11b-dihydro-6H-indeno[2,1-c]quinoline-3,6a,9,10-tetrol (47)
3-(3,4-dimethoxybenzyl)-7-methoxy-3,4-dihydro-2H-chromene-3,4-diol

### General Experimental Procedures

LC-MS conditions and NMR conditions: LC-MS was done using Luma-C18 column with mobile phase containing ammonium acetate in water and acetonitrile, and Zorbax SB column with mobile phase containing 0.1% ethyl acetate in water and acetonitrile. A flow rate of 1.0 ml/min. is maintained and 4.00 μl of the sample are injected for each run. ¹H NMR at 400 MHZ in DMSO, CD3OD and CDCl3 are done on the Varion NMR instrument to arrive at the chemical composition of the products formed.

Absorbance Spectra and Fluorescence Spectra conditions for compound+DNA/RNA/Protein Methods of experimentation: An Excitation Max: 488 nm, Emission Max: 560 nm, Band width: 488-600 nm on Alexa Fluor 488 channel were used for flow cytometry and microscopic determinations TECAN spectrofluorimeter.

### General Procedure for Recording Absorbance and Fluorescence Spectra:

1 mg/ml stock solutions of all the compounds were prepared in DMSO. Few insoluble compounds were then dissolved in non-polar solvent. 10 ul (1 mg/ml) of each of the compounds were used to do an absorbance scan in the TECAN nano reader and the graphs were plotted.

The absorbance peaks for each of the compounds were tabulated and further used as excitation wavelength for fluorescence scans. Initially an absorbance scan was performed to identify the excitation maxima after which the excitation wavelength was used to obtain the emission maxima/fluorescence plots. Once the excitation and emission maxima were known, different concentrations of the compounds were used starting from 10 ng to saturating concentrations to obtain fluorescent spectra. Similarly, a fixed concentration of the compound was used to which an increasing concentration of DNA/RNA was added until saturation to find the nucleic acid binding properties of the dyes and compounds. In addition, a fixed concentration of DNA/RNA was used, to which increasing concentrations of the compounds were added until saturation. The data obtained was plotted and used for calculating binding constants and determining the binding ability of the dyes and compounds.

General experimental procedures-assay methods-gel-electrophoresis, PCR, Staining methods: Microscopy & Flow Cytometry determinations were carried out using 1 mg/ml of the compound using PBS buffer for Cell Permeability and Fluorescence Excitation/Emission studies at 490 nm/560 nm at Room Temperature conditions.

Agarose gel electrophoresis is used to separate mixed populations of macromolecules such as DNA, RNA or proteins in a matrix of agarose. It is used to determine the approximate length of a DNA, RNA or PCR fragment by running them on an agarose gel alongside a DNA ladder. Method involved 12 RNA samples with control RNA and one DNA sample with control DNA run on the agarose gel alongside the DNA ladder and the bands were visualized under UV transilluminator.

The invention is further illustrated by the following examples which in no way should be construed as being further limiting. One skilled in the art will readily appreciate that the specific methods and results described are merely illustrative. Structures of the compounds as well as the final compounds were confirmed by nuclear magnetic resonance spectra for proton (H NMR) and LCMS.

The compounds of the present disclosure are prepared using the reactions and techniques described below, together with conventional techniques known to those skilled in the art of organic synthesis, or variations thereon as appreciated by those skilled in the art.

The reactions are performed in solvents appropriate to the reagents and materials employed and are suitable for the transformations being affected. Preferred methods include, but not limited to those described below, where all symbols are as defined earlier and otherwise defined below.

### EXAMPLES

The present invention is further elaborated with help of the following examples. However, these examples should not be construed to limit the scope of the invention.

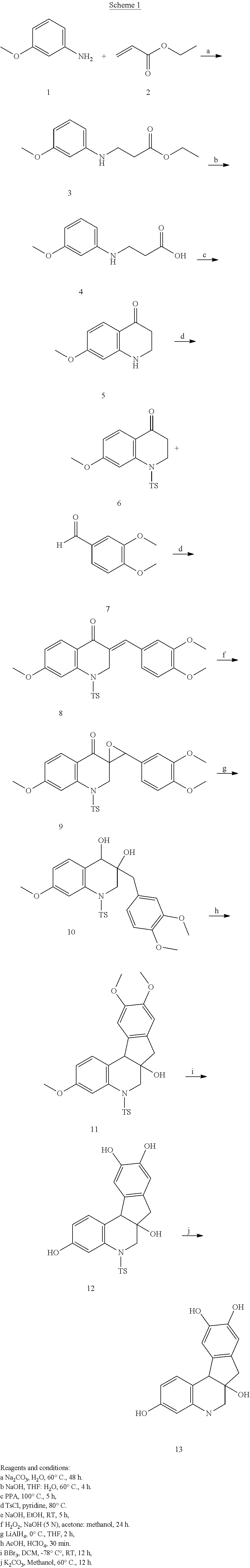

### Experimental Procedure:

### Synthesis of Ethyl 3-(3-methoxyanilino)propanoate (3)

To a stirred solution of meta-anisidine (20 g, 0.162 mol) and ethyl acrylate (19 ml, 0.178 mol) in water (100 ml) was added Na₂CO₃ (34.3 g, 0.3252 mol) and the resulting mixture was stirred at 80° C. for further 48 h. Reaction mixture was poured in water (300 ml), extracted with ethyl acetate (200 ml×2). Organic layer was washed with water (100 ml) and the organic layer was separated, dried over Na₂SO₄, evaporated under reducing pressure. Crude compound was purified by column chromatography by eluting with 3% ethyl acetate in hexane to afford the desired product 8 g (yield 22%) as a brown liquid.

¹HNMR (400 MHz, DMSO-d6): 6.96 (t, J=8.0 Hz, 1H), 6.16-6.11 (m, 3H), 5.62 (s, 1H), 4.06 (q, J=7.2 Hz, 2H), 3.66 (s, 3 h), 3.27 (q, J=6.8 Hz, 2H), 2.54 (t, J=6.54, 2H), 1.18 (t, J=6.8 Hz, 3H), LCMS: 224(M+1).

### Synthesis of 3-(3-methoxyanilino)propanoic acid (4)

To a stirred solution of Ethyl 3-(3-methoxyanilino)propanoate (8 g, 0.0358 mol) in THF/water (60 ml, 1:1 ratio) was added NaOH (6N, 20 ml) at RT, and the resulting mixture was stirred at 60° C. 12 h. After completion of reaction, crude reaction mixture was acidified with citric acid solution until pH 5 and further extracted with ethyl acetate (200 ml×2). Organic layer was separated dried over Na₂SO₄, evaporated under reducing pressure. Crude weight, 6.5 g (Yield 92%) as a brown color liquid. Crude compound was taken as such for the next step without purification. LCMS: 196(M+1).

### Synthesis of 7-methoxy-2,3-dihydro-1H-quinolin-4-one (5)

To a round bottom flask, PPA (20 g) was heated to 90° C. with mechanical stirring. 3-(3-methoxyanilino) propanoic acid (4) (6.5 g, 0.0333 mol) was added in one portion, and the mixture was stirred for 2 h. After cooling to 60° C., ice (50 g) was added and the reaction mixture was stirred until the reaction was complete (15 min). Then the product was extracted with EtOAc (4×100 mL). The combined organic layers were washed with H₂O (100 mL) and aq NaOH (5%, 15 mL). Then the organic layer was washed with H₂O (100 mL) and with brine (100 mL), dried over MgSO4, and concentrated under reduced pressure. The resulting brown oil was purified by flash column chromatography (EtOAc-hexane 1:1) to afford the title compound. Yellow solid, 3.55 g (Yield 60%).

1HNMR (400 MHz, DMSO-d6): 7.51 (d, J=8.8 Hz, 1H), 6.75 (s, 1H), 6.21-6.17 (m, 2H), 3.73 (s, 3H), 3.41-3.37 (m, 2H), 2.44 (t, J=6.8, 2H). LCMS: 178(M+1).

### Synthesis of 7-methoxy-1-(p-tolylsulfonyl)-2,3-dihydroquinolin-4-one (6)

Procedure: To a stirred solution of 7-methoxy-2,3-dihydro-1H-quinolin-4-one (3.5 g, 0.0197 mol) in pyridine (50 ml,) was added para-toluenesulphonyl chloride (TsCl) (4.5 g, 0.0237 mol) portion wise at 0° C. Further resulting suspension was heated to 80° C. for 12 h. After completion of the reaction, excess pyridine was removed by rotary evaporator; crude compound was dissolved in water and extracted by ethyl acetate (2×100 mL). The combined organic layers were washed with brine (100 mL), dried over Na₂SO₄, and concentrated under reduced pressure. The resulting brown oil was purified by flash column chromatography (EtOAc-hexane 1:1) to afford the title compound. Off-white solid, 3.22 g (Yield 49%).

1HNMR (400 MHz, DMSO-d6): 7.90 (d, J=8.8 Hz, 1H), 7.58 (d, J=8.0 Hz, 2H), 7.37 (d, J=2.4 Hz, 1H), 7.26-7.22 (m, 2H), 6.78 (dd, J=2.4 Hz, 1H), 4.21 (t, J=6.4 Hz, 2H), 3.90 (s, 3H), 2.38 (s, 3H), 2.31 (t, J=6.4 Hz, 2H), LCMS: 332(M+1).

### Synthesis of (3E)-3-[(3,4-dimethoxyphenyl)methylene]-7-methoxy-1-(p-tolylsulfonyl)-2H-quinolin-4-one (8)

To a stirred solution of 7-methoxy-1-(p-tolylsulfonyl)-2,3-dihydroquinolin-4-one (6) (3.2 g, 0.0096 mol) and 3,4 dimethoxy benzaldehyde (1.75 g, 0.0105 mol) in ethanol (100 ml,) was added NaOH (6N, 4 ml) at 0° C. Further, resulting mixture was stirred at rt for 1 h. Solid precipitation was filtered and washed with water (50 ml). Solid was dried under vacuum overnight. Yield 2.9 g (Yield 63%) off-white solid.

¹HNMR (400 MHz, DMSO-d₆): 7.91 (d, J=8.8 Hz, 1H), 7.37 (s, 1H), 7.31 (d, J=2.0 Hz, 1H), 7.12 (d, J=8.4 Hz, 2H), 6.99-6.97 (m, 4H), 6.90-6.88 (m, 2H), 5.09 (s, 2H), 3.98 (s, 3H), 3.97 (s, 3H), 3.94 (s, 3H), 2.33 (s, 3H). LCMS: 480(M+1).

### Synthesis of 3-[(3,4-dimethoxyphenyl)methyl]-3,7-dimethoxy-1-(p-tolylsulfonyl)-2H-quinolin-4-one (9)

To a stirred solution of (3E)-3-[(3,4-dimethoxyphenyl)methylene]-7-methoxy-1-(p-tolylsulfonyl)-2H-quinolin-4-one (8) (2.9 g, 0.00605 mol) in Acetone:methanol (50 ml, 1:1 ratio) was added 30% H₂O₂ in water (20 ml) at RT. Further, the reaction mixture was cooled to 0° C., added 5% NaOH in water, and the resulting mixture was stirred at rt for 12 h. Reaction mixture was diluted with 100 ml water, solid precipitate formed was filtered and dried by high vacuum to afford the desired product 2.5 g (83.6%) as a white color solid F.

¹HNMR (400 MHz, DMSO-d6): 7.81 (d, J=8.8 Hz, 1H), 7.47 (d, J=8.4 Hz, 2H), 7.35 (d, J=8.4 Hz, 2H), 7.14 (d, J=2 Hz, 1H), 7.06 (d, J=8.4 Hz, 1H), 7.0 (s, 1H), 6.97-6.92 (m, 2H), 4.5 (s, 1H), 4.2 (s, 1H), 3.8 (s, 3H), 3.79 (s, 3H), 3.78 (s, 3H), 2.35 (s, 3H), 2.32 (s, 1H). LCMS: 496(M+1).

### Synthesis of 3-[(3,4-dimethoxyphenyl)methyl]-7-methoxy-1-(p-tolylsulfonyl)-2,4-dihydroquinoline-3,4-diol (10)

To a stirred suspension of LAH (2.5 g, 0.00505 mol) in THE (50 ml), added drop wise 3-[(3,4-dimethoxyphenyl)methyl]-3,7-dimethoxy-1-(p-tolylsulfonyl)-2H-quinolin-4-one (9) (0.191 g, 0.00505 mol) at 0° C. under nitrogen atmosphere. Further, the resulting mixture was stirred at rt for 6 h. Reaction was monitored by TLC, after completion of the reaction, it was cooled to 0° C., quenched with saturated ammonium chloride (20 ml), added water (100 ml), extracted with ethyl acetate (250 ml×3). Organic layer was washed with a saturated brine solution (100 ml). Organic layer evaporated under reducing pressure. Crude was purified by prep HPLC method to afford the desired products 0.4 g (Yield 16%), off-white color solid.

¹HNMR (400 MHz, DMSO-d6): 7.47 (d, J=8.4 Hz, 2H), 7.28-7.23 (m, 3H), 7.07 (d, J=2 Hz, 1H), 6.90 (d, J=8 Hz, 1H), 6.80 (d, J=1.6 Hz, 1H), 6.74-6.66 (m, 2H), 5.39 (s, 1H), 4.59 (s, 1H), 3.87 (s, 1H), 3.76 (s, 2H), 3.72 (s, 3H), 3.69 (s, 3H), 3.68 (s, 3H), 2.70-2.67 (m, 2H), 2.32 (s, 3H). LCMS: 498(M−1).

### Synthesis of 3,9,10-trimethoxy-5-(p-tolylsulfonyl)-7,11b-dihydro-6H-indeno[2,1-c]quinolin-6a-ol (11)

To a stirred solution of 3-[(3,4-dimethoxyphenyl)methyl]-7-methoxy-1-(p-tolylsulfonyl)-2,4-dihydroquinoline-3,4-diol (0.4 g, 0.000801 mol) in acetic acid (5 ml,) was added per chloric acid (70%) (0.1 ml) at RT, and the resulting mixture was stirred at rt for 30 min. Reaction was monitored by TLC, after completion of the reaction, mixture was poured into saturated sodium bicarbonate solution slowly, precipitate was formed. It was filtered, washed with 10 ml water. Solid obtained was purified by Column chromatography by eluting with 50% Ethyl acetate in Hexane to afford the desired products 0.15 g (39.4%) as a white solid.

¹HNMR (400 MHz, CD3OD): 7.67 (d, J=8.4 Hz, 2H), 7.35 (d, J=8.4 Hz, 1H), 7.23 (d, J=8.4 Hz, 2H), 7.13 (d, J=3.6 Hz, 1H), 6.79 (s, 1H), 6.73 (dd, J=8.4 Hz, 1H), 6.64 (s, 1H), 4.05 (s, 1H), 3.90 (d, J=1.2 Hz, 1H), 3.79 (s, 3H), 3.73 (s, 1H), 3.69 (s, 6H), 3.15 (s, 2H), 2.35 (s 3H). LCMS: 482(M+1).

### Synthesis of 5-(p-tolylsulfonyl)-7,11b-dihydro-6H-indeno[2,1-c]quinoline-3,6a,9,10-tetrol (12)

Procedure: To a stirred solution of 3, 9,10-trimethoxy-5-(p-tolylsulfonyl)-7,11b-dihydro-6H-indeno[2,1-c]quinolin-6a-ol (11)(0.15 g, 0.000311 mol) in DCM (5 ml,) was added Boron Tribromide (1M in DCM) (3.1 ml, 0.00311 mol) at −78° C., and the resulting mixture was stirred at rt for 16 h. After completion of the reaction as monitored by TLC, it was quenched slowly with water at 0° C. Extracted with Ethyl acetate (100 ml×3). Crude was purified by PrepHPLC. Desire compound 44 mg (Yield 32%) as a red solid.

¹HNMR (400 MHz, CD3OD): 7.66 (d, J=8.4 Hz, 2H), 7.22 (d, J=8 Hz, 2H), 7.17 (d, J=8.4 Hz, 1H), 7.10 (d, J=2.4 Hz, 1H), 6.56 (s, 2H), 6.54 (d, J=2.4 Hz, 1H), 6.48 (s, 1H), 3.94 (d, J=6.4 Hz, 1H), 3.63 (d, J=13.2 Hz, 1H), 3.04 (d, J=6.4 Hz, 2H), 2.36 (s, 3H). LCMS: 438(M−1)

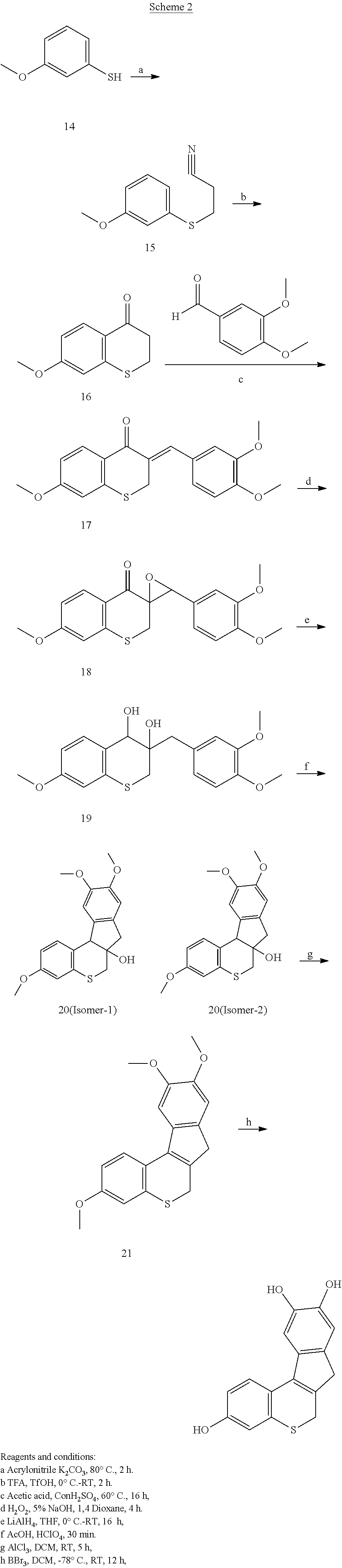

### Synthesis of 3-(3-methoxyphenyl)sulfanylpropanenitrile (15)

To a stirred solution of 3-methoxybenzenethiol (10.0 g, 0.0713 mol) in acrylonitrile (100 ml) was added K₂CO₃ (19.7 g, 0.4126 mol) and the resulting mixture was stirred at 80° C. for further 2 h. After completion of the reaction as monitored by TLC, the reaction solvent was evaporated and water was added (100 ml). The mixture was extracted with Ethyl acetate (100 ml×3). Organic layer was washed with water (100 ml) and evaporated under reducing pressure. Crude material was purified by Column chromatography by eluting with 20% Ethyl acetate in Hexane to afford the desired product 3-(3-methoxyphenyl) sulfanylpropanenitrile 13.5 g (97.3%) as a white color liquid.

¹HNMR (400 MHz, DMSO-d6): 7.282-7.241 (m, 1H), 6.961-6.935 (m, 2H), 6.825-6.798 (m, 1H), 3.762 (s, 3H), 3.257 (t, J=6.8 Hz, 2H), 2.787 (t, J=6.8 Hz, 2H).

### Synthesis of 7-methoxythiochroman-4-one (16)

To a stirred solution of 3-(3-methoxyphenyl) sulfanylpropanenitrile (13.5 g, 0.0695 mol) in Trifluoroacetic acid (21.3 ml, 0.278 mol) was added Triflic acid (9.2 ml, 0.0.1043 mol) at 0° C. and the resulting mixture was stirred at room temperature for further 2 h. After completion of the reaction as monitored by TLC, reaction was cooled to 0° C., quenched with water (100 ml) slowly, extracted with ethyl acetate (250 ml×3). Organic layer was washed with saturated NaHCO₃ solution (100 ml). The organic layer evaporated under reducing pressure. Crude was purified by column chromatography by eluting with 20% ethyl acetate in hexane to afford the 7-methoxythiochroman-4-one 13.5 g (97.3%) as a yellow color liquid.

¹HNMR (400 MHz, CDCl3): 8.085 (d, J=8.8 Hz, 1H), 6.74-6.69 (m, 2H), 3.84 (s, 3 h), 3.24-3.21 (m, 2H), 2.96-2.93 (m, 2H), LCMS: 195(M+1)

### Synthesis of (3Z)-3-[(3,4-dimethoxyphenyl)methylene]-7-methoxy-thiochroman-4-one (17)

To a stirred solution of 7-methoxythiochroman-4-one (13.0 g, 0.0669 mol) in Acetic acid (100 ml,) were added 3,4-dimethoxybenzaldehyde (13.34 g, 0.0803 mol), Con.H₂SO₄ (2 ml) at rt, and the resulting mixture was stirred at 60° C. for 16 h. After completion of the reaction as monitored by TLC, the reaction was cooled to rt, water was added (100 ml), extracted with ethyl acetate (250 ml×3). Organic layer was washed with saturated NaHCO₃ solution (100 ml). Organic layer was evaporated under reducing pressure. Crude compound was purified by column chromatography by eluting with 20% Ethyl acetate in Hexane to afford the (3Z)-3-[(3,4-dimethoxyphenyl)methylene]-7-methoxy-thiochroman-4-one 5.5 g (24%) as a white color solid.

¹HNMR (400 MHz, DMSO-d6): 8.007 (d, J=8.8 Hz, 1H), 7.56 (s, 1H), 7.11-7.06 (m, 3H), 6.93-6.86 (m, 2H), 4.28 (s, 2H), 3.84-3.80 (m, 9 h). LCMS: 343(M+1).

### Synthesis of 3-(3,4-dimethoxyphenyl)-7′-methoxy-spiro[oxirane-2,3′-thiochromane]-4′-one (18)

To a stirred solution of (3Z)-3-[(3,4-dimethoxyphenyl)methylene]-7-methoxy-thiochroman-4-one (5.5 g, 0.0160 mol) in 1,4-Dioxane (100 ml,) was added 30% H₂O₂ in water (10 ml) at rt, reaction was cooled to 0° C., added 5% NaOH in water, and the resulting mixture was stirred at rt for 4 h. After completion the reaction monitored by TLC, then added water (100 ml) to the reaction mixture and filtered the solid, and washed with 10 ml water, solid was fully dried under high vacuum to afford the 3-(3,4-dimethoxyphenyl)-7′-methoxy-spiro[oxirane-2,3′-thiochromane]-4′-one. 5.0 g (87.7%) as a white color solid.

¹HNMR (400 MHz, CDCl3): 8.122 (d, J=8.8 Hz, 1H), 7.011-6.98 (m, 1H), 6.944-6.940 (m, 1H), 6.89 (d, J=8.0 Hz, 1H), 6.79-6.76 (m, 1H), 6.715-6.709 (m, 1H), 4.37 (s, 1H), 3.909-3.901 (m, 6 h), 3.854 (s, 3H), 3.764 (d, J=13.2, Hz, 1H), 2.608 (d, J=13.6, Hz, 1H) LCMS: 359(M+1).

### Synthesis of 3-[(3,4-dimethoxyphenyl)methyl]-7-methoxy-thiochromane-3,4-diol (19)

To a stirred solution of 3-(3,4-dimethoxyphenyl)-7′-methoxy-spiro[oxirane-2,3′-thiochromane]-4′-one (3.0 g, 0.0083 mol in THE (100 ml,) was added LAH (0.477 g, 0.0125 mol) at 0° C. under nitrogen atmosphere, and the resulting mixture was stirred at RT for 6 h. After completion the reaction monitored by TLC, then reaction was cooled to 0° C., quenched with saturated ammonium chloride (5 ml), added water (100 ml), extracted with ethyl acetate (250 ml×3). Organic layer was washed with saturated brine solution (100 ml). Organic layer was evaporated under reduced pressure. Crude compound was purified by column chromatography by eluting with 50% ethyl acetate in hexane to afford the diastereomers of 3-[(3,4-dimethoxyphenyl)methyl]-7-methoxy-thiochromane-3,4-diol 1.9 g & 0.5 g as a white color solid.

LCMS: 361(M−1).

### Synthesis of 3,9,10-trimethoxy-6,7-dihydroindeno[2,1-c]thiochromene (20) (Isomer-1)

To a stirred solution of 3-[(3,4-dimethoxyphenyl)methyl]-7-methoxy-thiochromane-3,4-diol (0.500 g, 1.379 mmol) in acetic acid (2 ml,) was added perchloric acid (70%) (0.1 ml) at rt, and the resulting mixture was stirred at rt for 30 min. After completion the reaction was monitored, then quenched with saturated sodium bicarbonate solution slowly and the solid was filtered, washed with 10 ml water. Crude material was purified by column chromatography by eluting with 30% ethyl acetate in hexane to afford the 3,9,10-trimethoxy-7,11b-dihydro-6H-indeno[2,1-c]thiochromen-6a-ol 0.400 g (84%) as a white color solid.

¹HNMR (400 MHz, DMSO-d6): 7.383 (d, J=8.8 Hz, 1H), 6.814 (s, 1H), 6.769-6.728 (m, 2H), 6.651 (s, 1H), 5.434 (s, 1H), 4.077 (s, 1H), 3.710-3.702 (m, 6 h), 3.641 (s, 3H), 3.213 (d, J=16.4 Hz, 1H), 3.022 (d, J=16.4 Hz, 1H), 2.899 (d, J=13.2 Hz, 1H), 2.811 (d, J=13.6 Hz, 1H), HPLC: −99.97%, LCMS: 345(M+1), 327 (M+1) —H₂O.

### Synthesis of 3,9,10-trimethoxy-6,7-dihydroindeno[2,1-c]thiochromene:—(Isomer-2)

To a stirred solution of 3-[(3,4-dimethoxyphenyl)methyl]-7-methoxy-thiochromane-3,4-diol (0.500 g, 1.379 mmol) in acetic acid (2 ml,) was added per chloric acid (70%) (0.1 ml) at rt, and the resulting mixture was stirred at rt for 30 min. After completion the reaction was monitored by TLC, then quenched with saturated sodium bicarbonate solution slowly and filtered the solid, and washed with 10 ml water. Crude compound was purified by column chromatography by eluting with 30% ethyl acetate in hexane to afford the 3,9,10-trimethoxy-7,11b-dihydro-6H-indeno[2,1-c]thiochromen-6a-ol 0.400 g (84.3%) as a white color solid.

¹HNMR (400 MHz, DMSO-d6): 7.382 (d, J=8.4 Hz, 1H), 6.814 (s, 1H), 6.769-6.727 (m, 2H), 6.650 (s, 1H), 5.433 (s, 1H), 4.077 (s, 1H), 3.711-3.701 (m, 6 h), 3.641 (s, 3H), 3.212 (d, J=16.4 Hz, 1H), 3.022 (d, J=16.0 Hz, 1H), 2.899 (d, J=12.8 Hz, 1H), 2.811 (d, J=12.8 Hz, 1H), HPLC: −99.89%, LCMS: 345(M+1), 327 (M+1) —H₂O.

### Synthesis of 3,9,10-trimethoxy-6,7-dihydroindeno[2,1-c]thiochromene (21)

To a stirred solution of 3,9,10-trimethoxy-7,11b-dihydro-6H-indeno[2,1-c]thiochromen-6a-ol (0.100 g, 0.290 mmol) in DCM (5 ml,) was added AlCl₃ (0.155 g, 1.1627) at rt. and the resulting mixture was stirred at rt for 5 h. After completion of the reaction as monitored by TLC, the reaction was quenched slowly with 1N HCl (1 ml). Extracted with ethyl acetate (100 ml×3). Crude material was submitted for preparative HPLC, to afford 3,9,10-trimethoxy-6,7-dihydroindeno[2,1-c]thiochromene 0.030 g (31.2%) as a brown solid.

¹HNMR (400 MHz, CdCl3): 7.76 (d, J=8.8 Hz, 1H), 7.331 (s, 1H), 7.083 (s, 1H), 7.030-7.027 (m, 1H), 6.85-6.83 (m, 1H), 3.956-3.929 (m, 6 h), 3.844 (s, 3H), 3.762 (s, 2H), 3.512 (s, 2H). HPLC: −88.73%, LCMS: 327(M+1).

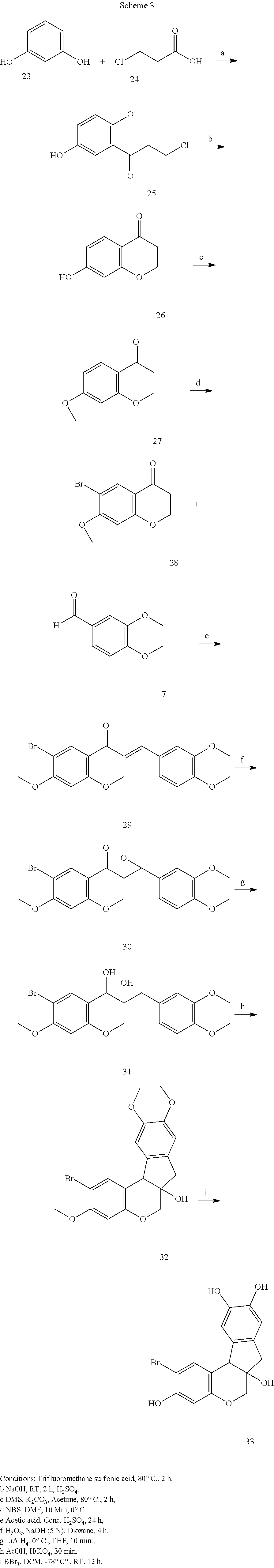

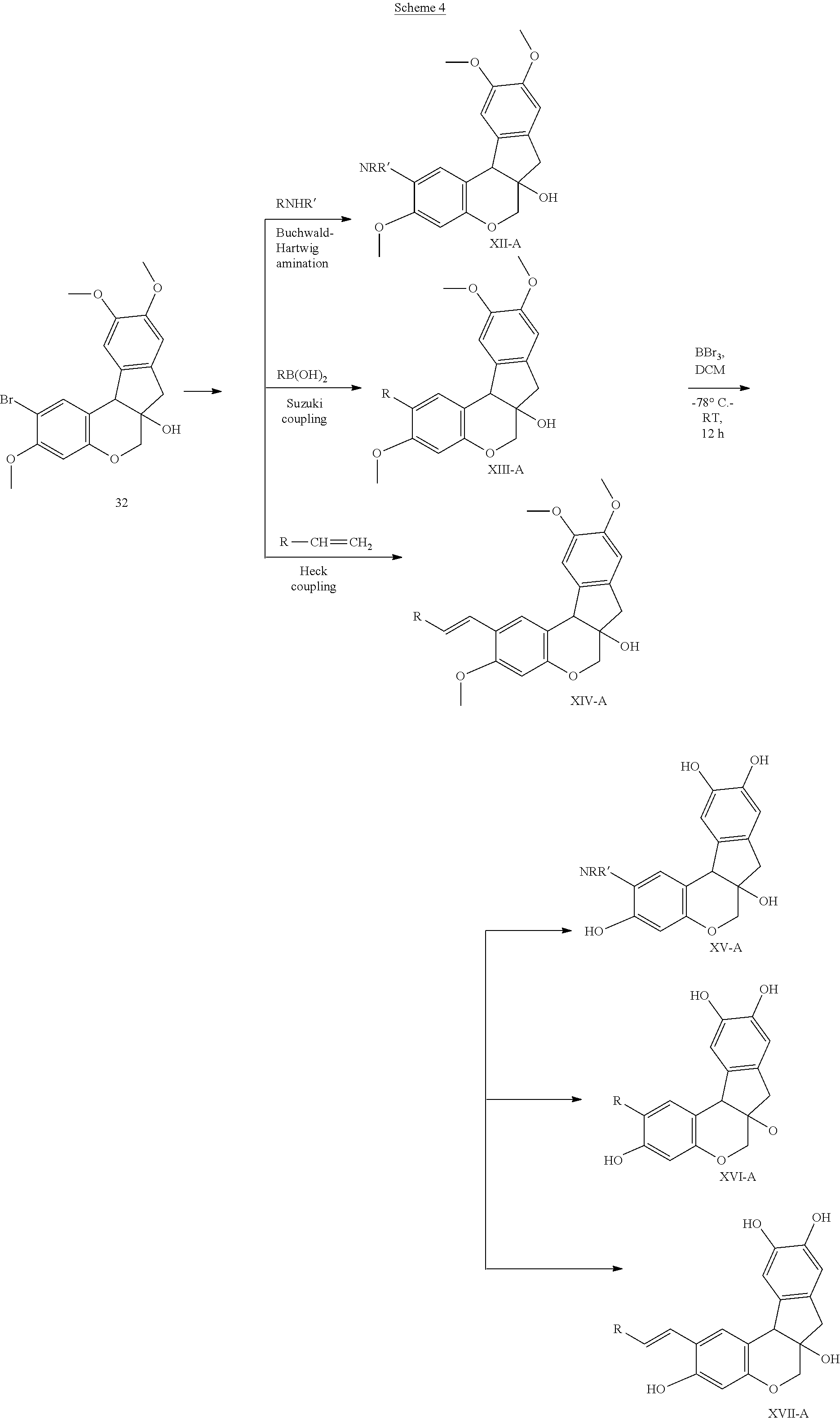

### Experimental Procedure:

### Synthesis of 3-chloro-1-(2,4-dihydroxyphenyl)propan-1-one (25)

To a stirred solution of resorcinol (1)(5 g, 0.0454 mol) in trifluoro methane sulphonic acid (14 ml) was added propionic acid (2)(4.92 g, 0.454 mol) and the resulting mixture was stirred at 80° C. for further 2 h. Reaction mixture was poured into water (300 ml), extracted with dichloromethane (200 ml×2). Organic layer was washed with water (100 ml) and the organic layer was separated and dried over Na₂SO₄, evaporated under reducing pressure to afford the desired product 4.1 g (yield 45%) as an orange solid.

¹HNMR (400 MHz, DMSO-d6): 12.52 (s, 1H), 7.62 (d, J=8.8 Hz, 1H), 6.43 (d, J=2.4 Hz, 1H), 6.41-6.39 (m, 1H), 3.9 (t, J=6.8 Hz, 2H), 3.4 (t, J=6.4 Hz, 2H), LCMS: 200(M+1)

### Synthesis of 7-hydroxy-2,3-dihydro-4H-chromen-4-one (26)

To a 3-chloro-1-(2,4-dihydroxyphenyl)propan-1-one (3) (4.1 g, 0.025 mol) was added NaOH (6N, 250 ml) at 5° C., and the resulting mixture was stirred for 2 h. After completion of reaction, crude reaction mixture was acidified with sulphuric acid solution until PH 2, and further extracted with ether (200 ml×2). Organic layer was separated and dried over Na₂SO₄, evaporated under reducing pressure. Crude weight, 3.8 g (Yield 92%) as a yellow solid. Crude compound was taken as such for the next step without purification.

¹HNMR (400 MHz, DMSO-d6): 7.69 (d, J=8.4 Hz, 1H), 6.48-6.45 (dd, J=8 Hz, 1H), 6.3 (d, J=2.4 Hz, 1H), 4.47 (t, J=6.4 Hz, 2H), 2.69 (t, J=6.4 Hz, 2H), LCMS: 164 (M+1)

### Synthesis of 7-methoxy-2,3-dihydro-4H-chromen-4-one (27)

To a stirred solution of 7-hydroxy-2,3-dihydro-4H-chromen-4-one (4)(3.8 g, 0.023 mol) in acetone was added dimethyl sulphate (6.73 ml, 0.06951 mol) and K₂CO₃ (6.39 g. 0.0463 mol) and the reaction mixture was stirred at 80° C. for 2 h. Then the product was extracted with EtOAc (4×100 mL). Then the organic layer was washed with H₂O (100 mL) and with brine (100 mL), dried over Na₂SO₄, and concentrated under reduced pressure. The resulting brown liquid was further taken to the next step. Yield 5.2 g (Yield 83%).

1HNMR (400 MHz, DMSO-d6): 7.83 (d, J=8.8 Hz, 1H), 6.59-6.56(dd, J=4 Hz, 1H), 6.41 (d, J=2.8 Hz, 1H), 4.52 (t, J=6 Hz, 2H), 2.75 (t, J=6 Hz, 2H), 3.83 (s, 1H). LCMS: 178(M+1)

### Synthesis of 6-bromo-7-methoxy-2,3-dihydro-4H-chromen-4-one (28)

To a stirred solution of 7-methoxy-2,3-dihydro-4H-chromen-4-one (5.2 g, 0.0292 mol) in anhydrous DMF (15 ml,) was added N-Bromosuccinamide (6.23 g, 0.035 mol) portion wise at 0° C. and stirred for 10 min. Solid precipitate was filtered and washed with water to obtain the desired product as a light brown solid. Yield (6 g 80%)

1HNMR (400 MHz, DMSO-d6): 7.83 (s, 1H), 6.75 (s, 1H), 2.738 (t, J=6.4 Hz, 3H), 4.54 (t, J=6.4 Hz, 3H), 3.90 (s, 1H). LCMS: 257(M+1)

### Synthesis of (3E)-6-bromo-3-(3,4-dimethoxybenzylidene)-7-methoxy-2,3-dihydro-4H-chromen-4-one (29)

To a stirred solution of 6-bromo-7-methoxy-2,3-dihydro-4H-chromen-4-one (6) (6 g, 0.0233 mol) and 3,4 dimethoxy benzaldehyde (7) (4.7 g, 0.028 mol) in acetic acid (20 ml,) was added con H₂SO₄(8 ml). Further, the resulting mixture was stirred at 80° C. for 24 h. The reaction mixture was quenched with water and extracted with Ethyl Acetate. The organic layer was dried over Na₂SO₄ and concentrated to get the crude and purified by column chromatography by eluting with 20% Ethyl acetate in Hexane to afford the desired product Yield 3.2 g (Yield 33%) yellow solid.

¹HNMR (400 MHz, DMSO-d₆): 8.19 (s, 1H), 7.8(s, 1H), 6.93 (d, J=8.4 Hz, 1H), 6.88 (d, J=1.6 Hz, 2H), 6.43 (s, 1H), 5.39 (d, J=1.6 Hz, 2H), 3.92 (t, J=8 Hz, 9H).

### Synthesis 3′-(3,4-dimethoxyphenyl)-7-methoxy-6-methyl-4H-spiro[chromene-3,2′-oxiran]-4-one (30)

To a stirred solution of (3E)-6-bromo-3-(3,4-dimethoxybenzylidene)-7-methoxy-2,3-dihydro-4H-chromen-4-one (8) (3.2 g, 0.0079 mol) in dioxane (10 ml) was added 30% H₂O₂ in water (10 ml) at RT. Further, the reaction mixture was cooled to 0° C., added 5% NaOH in water, and the resulting mixture was stirred at rt for 4 h. Reaction mixture was diluted with 100 ml water, solid precipitate formed was filtered and dried by high vacuum to afford the desired product 2.8 g (84%) as a white solid.

¹HNMR (400 MHz, DMSO-d6): 8.15 (s, 1H), 6.9 (s, 1H), 6.84 (s, 1H), 6.42 (s, 1H), 4.54 (t, J=8 Hz, 2H), 4.16 (d, J=12.4 Hz, 1H), 3.9(t, J=5.6, 9H).

### Synthesis of 3-(3,4-dimethoxybenzyl)-7-methoxy-6-methyl-3,4-dihydro-2H-chromene-3,4-diol (31)

To a stirred suspension of LAH (2.5 g, 0.00505 mol) in THE (50 ml), added drop 3′-(3,4-dimethoxyphenyl)-7-methoxy-6-methyl-4H-spiro[chromene-3,2′-oxiran]-4-one (9)(2.8 g, 0.0027 mol) at 0° C. under nitrogen atmosphere. Further, the resulting mixture was stirred at rt for 10 min. Reaction was monitored by TLC, after completion the reaction was cooled to 0° C., quenched with ice cold water, solid precipitate formed was filtered and dried under vacuum. Crude product was purified by Column chromatography by eluting with 25% Ethyl acetate in Hexane to afford the desired product Yield 600 mg (Yield 21%), off-white solid.

¹HNMR (400 MHz, DMSO-d6): 7.4 (s, 1H), 6.98 (d, J=8 Hz, 1H), 6.89-6.85 (m, 2H), 6.58 (s, 1H), 5.98 (d, J=4 Hz, 1H), 4.38 (s, 1H), 4.25 (d, J=4.8 Hz, 1H), 4.19(d, J=12.4, 1H), 3.79 (s, 3H), 3.77 (d, J=12 Hz, 6H).

### Synthesis of 2-bromo-3,9,10-trimethoxy-7,11b-dihydro-6H-indeno[2,1-c]chromen-6a-ol (32)

To a stirred solution of 3-(3,4-dimethoxybenzyl)-7-methoxy-6-methyl-3,4-dihydro-2H-chromene-3,4-diol (10)(0.4 g, 0.000801 mol) in acetic acid (5 ml,) was added per chloric acid (70%) (0.1 ml) at RT, and the resulting mixture was stirred at rt for 30 min. Reaction was monitored by TLC, after completion the reaction mixture was poured into saturated sodium bicarbonate solution slowly, precipitate formed was washed with 10 ml water. Solid obtained was purified by Column chromatography by eluting with 50% ethyl acetate in hexane to afford the desired products 0.16 g (25.5%) as an off brown solid.

¹HNMR (400 MHz, DMSO-d6): 7.7 (s, 1H), 6.93 (s, 1H), 6.81 (s, 1H), 6.57 (s, 1H), 5.42 (s, 1H), 3.98 (s, 1H), 3.91 (d, J=10.8 Hz, 1H), 3.76 (s, 1H), 3.74 (s, 1H), 3.7 (d, J=5.6 Hz, 6H), 2.9 (d, J=8.4 Hz, 2H). LCMS: 407(M−1).

### Synthesis of 2-bromo-7,11b-dihydro-6H-indeno[2,1-c]chromene-3,6a,9,10-tetrol (33)

To a stirred solution of 2-bromo-3,9,10-trimethoxy-7,11b-dihydro-6H-indeno[2,1-c]chromen-6a-ol (11)(0.15 g, 0.000311 mol) in DCM (5 ml,) was added boron tribromide (1M in DCM) (3.1 ml, 0.00311 mol) at −78° C., and the resulting mixture was stirred at rt for 16 h. After completion the reaction was monitored by TLC, then quenched slowly with water at 0° C. Reaction mixture was extracted with ethyl acetate (100 ml×3). Crude material was purified by PrepHPLC. Desired compound was obtained. Yield 40 mg (Yield 57%)

¹HNMR (400 MHz, DMSO-d6): 10.08 (s, 1H), 8.73 (s, 1H), 8.61 (s, 1H), 7.43 (s, 1H), 6.62 (s, 1H), 6.53 (s, 1H), 6.41 (s, 1H), 5.33 (s, 1H), 3.85 (d, J=7.2 Hz, 1H), 3.56 (d, J=11.2 Hz, 1H), 3.16 (s, 3H), 2.86 (d, J=15.6 Hz, 1H), 2.68 (d, J=15.2 Hz, 1H). LCMS: 365(M+1).

### Synthesis of 3,9,10-trimethoxy-2-phenyl-7,11b-dihydro-6H-indeno[2,1-c]chromen-6a-ol (34)

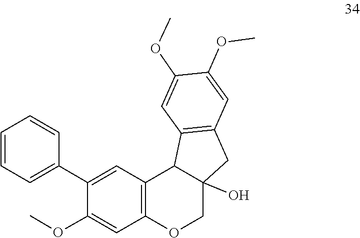 [SMILES: COc1cc2c(cc1OC)C1c3cc(-c4ccccc4)c(OC)cc3OCC1(O)C2]

To a stirred solution of 33 {2-bromo-3,9,10-trimethoxy-7,11b-dihydro-6H-indeno[2,1-c]chromen-6a-ol (0.1 g, 0.00024 mol)} in Dioxane:Water (5 ml) was added phenyl boronic acid (0.045 g, 0.000368 mol), K₂CO₃ (0.1 g, 0.000735 mol), and the resulting reaction mixture was degasified with argon for 15 min. Further, Pd(dppf)Cl₂. DCM (20 mg, 0.000025 mol) was added and the reaction mixture was heated to 110° C., for 16 h. Completion of the reaction monitored by TLC. Reaction mixture was filtered through a celite bed, washed with ethyl acetate. Organic layer was washed with a saturated brine solution (20 ml). Organic layer evaporated under reducing pressure. Crude was purified by Column chromatography by eluting with 40% Ethyl acetate in Hexane to afford the product 50 mg (50%) as a white color solid.

¹HNMR (400 MHz, CDCl₃): 7.8 (d, J=6.8 Hz, 2H), 7.47 (m, 3H), 6.98 (s, 1H), 6.89 (s, 2H), 6.53 (s, 1H), 5.88 (s, 1H), 4.59 (s, 1H), 4.35 (d, J=11.2 Hz, 1H), 4.15 (d, J=10.8 Hz, 1H), 3.88 (s, 3H), 3.85 (s, 3H), 3.85 (s, 3H), 3.74 (s, 3H). LCMS: 404 (M).

### Synthesis of 2-phenyl-7,11b-dihydro-6H-indeno[2,1-c]chromene-3,6a,9,10-tetrol (35)

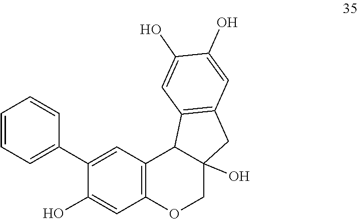 [SMILES: Oc1cc2c(cc1O)C1c3cc(-c4ccccc4)c(O)cc3OCC1(O)C2]

To a stirred solution of 3,9,10-trimethoxy-2-phenyl-7,11b-dihydro-6H-indeno[2,1-c]chromen-6a-ol (50 mg, 0.000123 mol) in DCM (10 ml,) was added BBr₃ (0.74 ml, 0.000742 mol) at −78° C. Further, the resulting mixture was stirred at rt for 2 h. After completion the reaction was monitored by LCMS, then reaction mixture was quenched slowly with water under cooling condition. Reaction mixture was extracted with DCM (20 ml×3). Crude was submitted for PrepHPLC, to offered title product 5 mg (11%) as a violet color solid.

¹HNMR (400 MHz, CD₃OD): 7.57 (d, J=1.2 Hz, 2H), 7.36 (t, J=7.2 Hz, 2H), 7.25 (t, J=5.2 Hz, 2H), 6.82 (s, 1H), 6.78 (s, 1H), 6.4 (s, 1H), 4.47 (s, 1H), 4.10 (s, 1H), 3.67 (d, J=11.2 Hz, 3H), 3.50 (s, 1H), LCMS: 361 (M−1)

### Synthesis of 2-(4-fluorophenyl)-3,9,10-trimethoxy-7,11b-dihydro-6H-indeno[2,1-c]chromen-6a-ol (36)

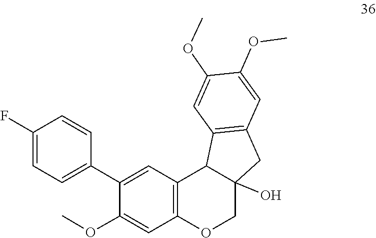 [SMILES: COc1cc2c(cc1OC)C1c3cc(-c4ccc(F)cc4)c(OC)cc3OCC1(O)C2]

To a stirred solution of 2-bromo-3,9,10-trimethoxy-7,11b-dihydro-6H-indeno[2,1-c]chromen-6a-ol (0.2 g, 0.000495 mol) in dioxane:water (8 ml) was added 4-fluoro phenyl boronic acid (0.1 g, 0.000737 mol), K₂CO₃ (0.20 g, 0.00147 mol), and the resulting reaction mixture was degasified with argon for 15 min. Further, Pd(dppf)Cl₂. DCM (40 mg, 0.0000491 mol) was added and the reaction mixture was heated to 110° C., for 16 h. Completion of the reaction monitored by TLC. Reaction mixture was filtered through a celite bed, washed with ethyl acetate. Organic layer was washed with a saturated brine solution (20 ml). The organic layer was evaporated under reducing pressure. Crude was purified by Column chromatography by eluting with 30% Ethyl acetate in Hexane to afford the product 80 mg (44%) as a grey color solid.

¹HNMR (400 MHz, CDCl3): 7.48 (m, 2H), 7.28 (s, 1H), 7.10 (t, J=9.2 Hz, 2H), 6.98 (s, 1H), 6.81 (s, 1H), 6.58 (s, 1H), 5.71 (s, 1H), 4.28 (s, 1H), 4.19 (dd, J=11.6 Hz, 1H), 3.88 (s, 1H), 3.86 (s, 3H), 3.85 (s, 3H), 3.7 (s, 3H), 3.74 (s, 1H), 2.67 (s, 1H), 2.18 (s, 3H). LCMS: 421 (M−1).

### Synthesis of 2-(4-fluorophenyl)-7,11b-dihydro-6H-indeno[2,1-c]chromene-3,6a,9,10-tetrol (37)

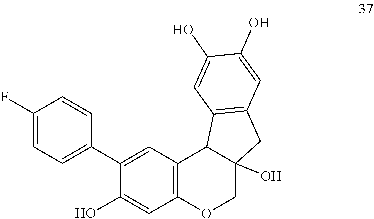 [SMILES: Oc1cc2c(cc1O)C1c3cc(-c4ccc(F)cc4)c(O)cc3OCC1(O)C2]

To a stirred solution of 2-(4-fluorophenyl)-3,9,10-trimethoxy-7,11b-dihydro-6H-indeno[2,1-c]chromen-6a-ol (25 mg, 0.0000434 mol) in DCM (10 ml,) was added BBr₃ (0.6 ml, 0.000260 mol) at −78° C. Further, the resulting mixture was stirred at rt for 2 h. After completion the reaction was monitored by LCMS, then reaction mixture was quenched slowly with water under cooling condition. Reaction mixture was extracted with DCM (20 ml×3). Crude was submitted for PrepHPLC, to offered title product 8 mg (11%) as a violet color solid.

¹HNMR (400 MHz, CD₃OD): 7.57 (t, J=8.4 Hz, 2H), 7.24 (s, 1H), 7.09 (t, J=8.8 Hz, 2H), 6.80 (d, J=15.2 Hz, 2H), 6.4 (s, 1H), 4.52 (s, 1H), 4.47 (s, 1H), 4.10 (s, 1H), 3.95 (d, J=11.6 Hz, 1H), 3.67 (d, J=3.2 Hz, 1H), LCMS: 379 (M−1)⁺

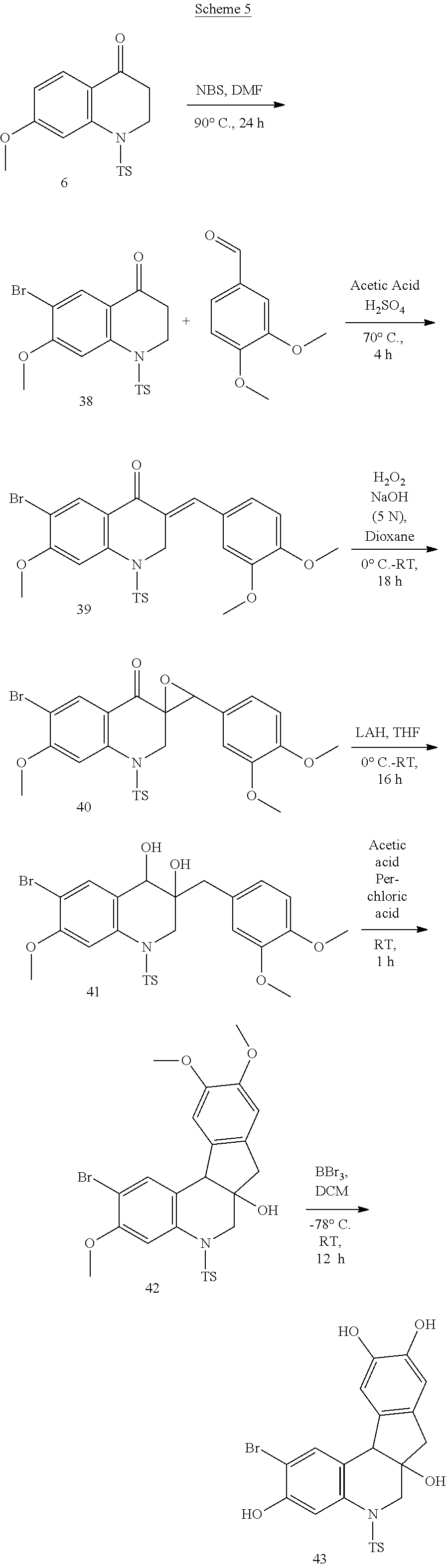

### Synthesis of 6-bromo-7-methoxy-1-sulfanyl-2,3-dihydroquinolin-4-one (38)

Procedure: To a solution of 7-methoxy-1-(p-tolylsulfonyl)-2,3-dihydroquinolin-4-one (4.6 g, 0.0139 mol) in DMF (40 ml) was added N bromosuccinimide (3 g, 0.0167 mol) portion wise at 0° C., it was then heated at 90° C. for 24 h. After the completion of reaction, it was quenched with water, thus solid precipitate was filtered and washed with water. Solid was dried under vacuum, overnight. Off-white Solid, Yield 5 g (Yield 87%).

¹HNMR (400 MHz, CDCl₃): 8.11 (s, 1H), 7.56 (d, J=8.4 Hz, 2H), 7.4 (s, 1H), 7.24 (s, 2H), 4.21 (t, J=6.4 Hz, 2H), 4.01 (s, 3H), 2.4 (s, 3H), 2.26 (t, J=6.4 Hz, 2H), LCMS: 412.13 (M+2)⁺

### Synthesis of (3E)-6-bromo-3-[(3,4-dimethoxyphenyl)methylene]-7-methoxy-1-(p-tolylsulfonyl)-2H-quinolin-4-one (39)

To a stirred solution of 6-bromo-7-methoxy-1-sulfanyl-2,3-dihydroquinolin-4-one (5 g, 0.0122 mol) and 3,4 dimethoxy benzaldehyde (2.02 g, 0.0122 mol) in Acetic acid (100 ml,) was added Sulphuric acid (2.5 ml) at 0° C. Further, the resulting mixture was heated at 70° C. for 4 h. After the completion of the reaction, it was quenched with water. Solid precipitate was filtered and washed with water (50 ml). Solid was dried under vacuum and purified by flash column chromatography (EtOAc-hexane 1:1) to afford the title compound, Yellow solid, 4.5 g (Yield 66%).

¹HNMR (400 MHz, CDCl₃): 8.14 (s, 1H), 7.38 (s, 1H), 7.33 (s, 1H), 7.1 (d, J=8.4 Hz, 2H), 7 (m, 4H), 6.89 (s, 1H), 5.1 (s, 1H), 4.05 (s, 3H), 3.98 (s, 3H), 3.97 (s, 3H), 2.3 (s, 3H). LCMS: 560.1 (M+2)⁺

### Synthesis of 6-bromo-3′-(3,4-dimethoxyphenyl)-7-methoxy-1-(p-tolylsulfonyl)spiro[2H-quinoline-3,2′-oxirane]-4-one (40)

Procedure: To a stirred solution of (3E)-6-bromo-3-[(3,4-dimethoxyphenyl)methylene]-7-methoxy-1-(p-tolylsulfonyl)-2H-quinolin-4-one (4.5 g, 0.0081 mol) in Dioxane (100 ml) was added 33% H₂O₂ in water (40 ml) at RT. Further, the reaction mixture was cooled to 0° C., added 5% NaOH in water, and the resulting mixture was stirred at room temperature for 18 h. Reaction mixture was diluted with 200 ml water, extracted with ethyl acetate. Organic layer was concentrated under reduced pressure. It was then recrystallized using water. Thus, solid precipitate formed was filtered and dried under high vacuum to afford the desired product Yellow Solid 4 g (86%).

¹HNMR (400 MHz, CDCl3): 8.08 (s, 1H), 7.47 (d, J=8.4 Hz, 2H), 7.38 (s, 1H), 7.21 (d, J=8 Hz, 2H), 6.9 (m, 2H), 6.8 (s, 1H), 4.51 (s, 1H), 4.5 (s, 1H), 4.28 (m, 1H), 4.01 (s, 3H), 3.97 (s, 1H), 3.93 (s, 3H), 3.92 (s, 3H), 2.39 (s, 1H). LCMS: 576(M+2)⁺

### Synthesis of 6-bromo-3-[(3,4-dimethoxyphenyl)methyl]-7-methoxy-1-(p-tolylsulfonyl)-2,4-dihydroquinoline-3,4-diol (41)

To a stirred solution of 6-bromo-3′-(3,4-dimethoxyphenyl)-7-methoxy-1-(p-tolylsulfonyl)spiro[2H-quinoline-3,2′-oxirane]-4-one (1.0 g, 0.001742 mol) in THE (100 ml,) was added LAH (0.477 g, 0.00209 mol) portion wise at 0° C. under nitrogen atmosphere, and the resulting mixture was stirred at rt for 10 min. Completion of the reaction monitored by TLC, then reaction was cooled to 0° C., quenched with saturated ammonium chloride (20 ml), added water (100 ml), extracted with ethyl acetate (100 ml×2). Organic layer was washed with a saturated brine solution (100 ml). Organic layer was evaporated under reducing pressure. Crude was taken as such for the next step without further purification. Crude yield, 0.9 g. LCMS: 579 (M+1).

### Synthesis of 2-bromo-3,9,10-trimethoxy-5-(p-tolylsulfonyl)-7,11b-dihydro-6H-indeno[2,1-c]quinolin-6a-ol (42)

To a stirred solution of 6-bromo-3-[(3,4-dimethoxyphenyl)methyl]-7-methoxy-1-(p-tolylsulfonyl)-2,4-dihydroquinoline-3,4-diol (0.9 g, 0.00155 mol) in acetic acid (5 ml,) was added per chloric acid (70%) (0.1 ml) at rt, and the resulting mixture was stirred at rt for 30 min. Completion of the reaction monitored by TLC, then quenched with saturated sodium bicarbonate solution slowly and filtered the solid, and washed with 50 ml water. Crude was purified by Column chromatography by eluting with 30% Ethyl acetate in Hexane to afford the product 0.2 g (22.9%) as a grey color solid.

¹HNMR (400 MHz, CD₃OD): 7.68 (d, J=8.4 Hz, 2H), 7.59 (s, 1H), 7.25 (d, J=1.6 Hz 3H), 6.78 (s, 1H), 6.59 (s, 1H), 4.01 (t, J=1.6 Hz, 2H), 3.78 (s, 3H), 3.74 (s, 3H), 3.70 (s, 3H), 3.69 (d, J=2.4 Hz, 1H), 3.12 (s, 1H), LCMS: 560 (M)

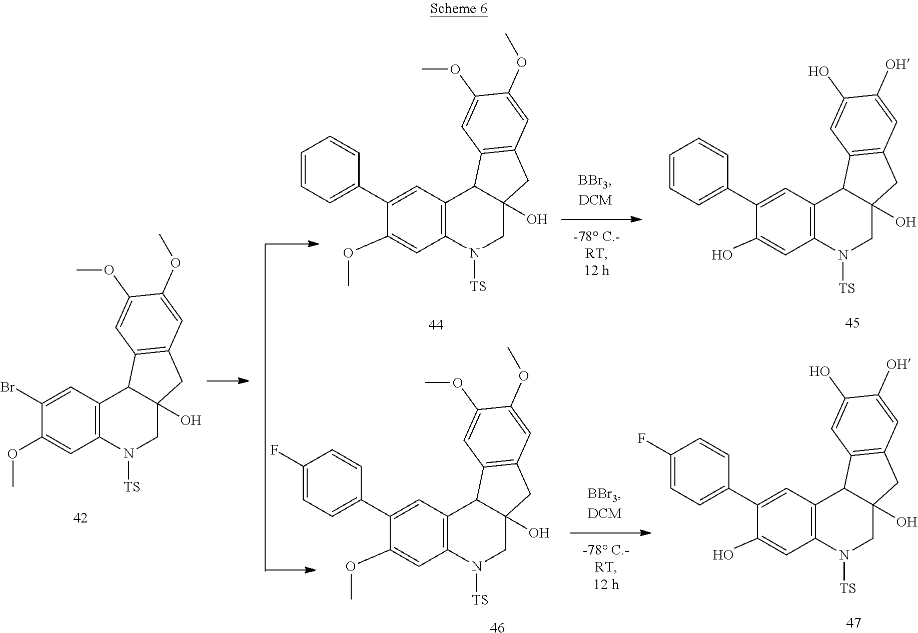

### Synthesis of 3,9,10-trimethoxy-2-phenyl-5-(p-tolylsulfonyl)-7,11b-dihydro-6H-indeno[2,1-c]quinolin-6a-ol (44)

To a stirred solution of comp 42(0.14 g, 0.00025 mol) in Dioxane:Water (5 ml) was added phenyl boronic acid (0.046 g, 0.000375 mol), K₂CO₃ (0.1 g, 0.00075 mol), and the resulting reaction mixture was degasified with argon for 15 min. Further, Pd(dppf)Cl₂. DCM (20 mg, 0.000025 mol) was added and the reaction mixture was heated to 110° C., for 16 h. Completion of the reaction monitored by TLC. Reaction mixture was filtered through a celite bed, washed with ethyl acetate. Organic layer was washed with a saturated brine solution (20 ml). Organic layer was evaporated under reducing pressure. Crude was purified by Column chromatography by eluting with 40% Ethyl acetate in Hexane to afford the product 60 mg (43%) as a grey color solid.

¹HNMR (400 MHz, CDCl₃): 7.7 (d, J=Hz, 2H), 7.4 (d, J=7.6 Hz, 3H), 7.34 (t, J=7.2 Hz, 2H), 7.28 (d, J=5.2 Hz, 2H), 7.21 (s, 1H), 7.14 (s, 1H), 6.85 (s, 1H), 6.58 (s, 1H), 5.43 (s, 1H), 4.4 (d, J=14 Hz, 1H), 4.0 (s, 1H), 3.8 (s, 3H), 3.7 (S, 3H), 3.66 (s, 3H), 3.28 (d, J=14 Hz, 1H), 3.0 (s, 1H), 2.8 (s, 1H), 2.3 (s, 3H). LCMS: 556(M).

### Synthesis of 3,9,10-trimethoxy-2-phenyl-5-(p-tolylsulfonyl)-7,11b-dihydro-6H-indeno[2,1-c]quinolin-6a-ol (45)

To a stirred solution of 3,9,10-trimethoxy-2-phenyl-5-(p-tolylsulfonyl)-7,11b -dihydro-6H-indeno[2,1-c]quinolin-6a-ol (44) (60 mg, 0.00010 mol) in DCM (10 ml,) was added BBr₃ (0.6 ml, 0.000642 mol) at −78° C. Further, the resulting mixture was stirred at rt for 2 h. After completion the reaction was monitored by LCMS, then reaction mixture was quenched slowly with water under cooling condition. Reaction mixture was extracted with DCM (20 ml×3). Crude was submitted for PrepHPLC, to offer the title product 15 mg (27%) as a light brown color solid.

¹HNMR (400 MHz, CD₃OD): 7.7 (d, J=7.6 Hz, 2H), 7.58 (d, J=8 Hz, 2H), 7.37 (t, J=7.2 Hz, 3H), 7.29 (m, 5H), 6.58 (d, J=8 Hz, 2H), 4.0 (m, 2H), 3.62 (d, J=13.2 Hz, 1H), 3.0 (d, J=15.2 Hz, 1H), 2.3 (s, 3H), 2.899 (d, J=13.2 Hz, 1H), 2.811 (d, J=13.6 Hz, 1H), LCMS: 516 (M+1).

### Synthesis of 2-(4-fluorophenyl)-3,9,10-trimethoxy-5-(p-tolylsulfonyl)-7,11b-dihydro-6H-indeno[2,1-c]quinolin-6a-ol (46)

To a stirred solution of compound 42(0.3 g, 0.000536 mol) in Dioxane:Water (8 ml) was added 4-fluoro phenyl boronic acid (0.11 g, 0.000804 mol), K₂CO₃ (0.22 g, 0.00160 mol), and the resulting reaction mixture was degasified with argon for 15 min. further, Pd(dppf)Cl₂. DCM (44 mg, 0.0000536 mol) was added and the reaction mixture was heated to 110° C., for 16 h. Completion of the reaction monitored by TLC. Reaction mixture was filtered through a celite bed, washed with ethyl acetate. Organic layer was washed with a saturated brine solution (20 ml). Organic layer was evaporated under reducing pressure. Crude was purified by Column chromatography by eluting with 40% Ethyl acetate in Hexane to afford the product 25 mg (8.33%) as a grey color solid.

¹HNMR (400 MHz, CDCl₃): 7.7 (d, J=8.4 Hz, 2H), 7.52 (m, 2H), 7.41 (s, 1H), 7.32 (s, 1H), 7.25 (d, J=8 Hz, 1H), 7.1 (t, J=8.8 Hz, 2H), 6.94 (s, 1H), 6.53 (s, 1H), 5.24 (s, 1H), 4.49 (d, J=13.6 Hz, 1H), 3.8 (s, 3H), 3.71 (s, 3H), 3.67 (s, 3H), 2.3 (s, 3H). LCMS: 575 (M)⁺.

### Synthesis of 2-(4-fluorophenyl)-5-(p-tolylsulfonyl)-7,11b-dihydro-6H-indeno[2,1-c]quinoline-3,6a,9,10-tetrol (47)

To a stirred solution of 3,9,10-trimethoxy-7,11b-dihydro-6H-indeno[2,1-c]thiochromen-6a-ol (46) (25 mg, 0.0000434 mol) in DCM (10 ml,) was added BBr₃ (0.6 ml, 0.000260 mol) at −78° C. Further, the resulting mixture was stirred at rt for 2 h. After completion the reaction was monitored by LCMS, then the reaction mixture was quenched slowly with water under cooling condition. Reaction mixture was extracted with DCM (20 ml×3). Crude was purified by PrepHPLC. LCMS: 575 (M)⁺.

TABLE 1
Prophetic Examples

| No | Structure |
|---|---|
| 1 | 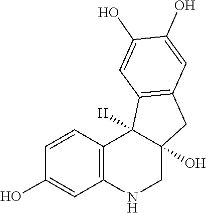 [SMILES: Oc1ccc2c(c1)NC[C@]1(O)Cc3cc(O)c(O)cc3[C@H]21] |
| 2 | 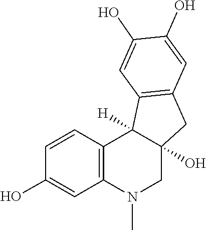 [SMILES: CN1C[C@]2(O)Cc3cc(O)c(O)cc3[C@@H]2c2ccc(O)cc21] |
| 3 | 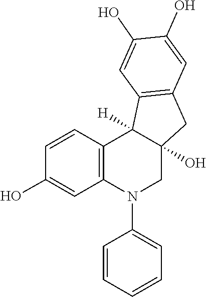 [SMILES: Oc1ccc2c(c1)N(c1ccccc1)C[C@]1(O)Cc3cc(O)c(O)cc3[C@H]21] |
| 4 | 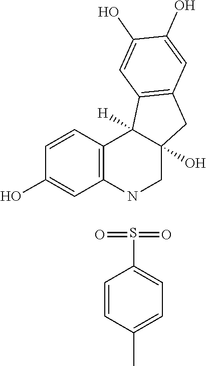 |
| 5 | 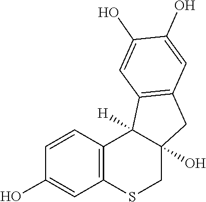 [SMILES: Oc1ccc2c(c1)SC[C@]1(O)Cc3cc(O)c(O)cc3[C@H]21] |
| 6 | 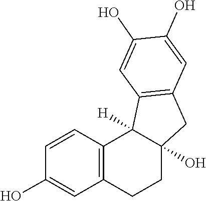 [SMILES: Oc1ccc2c(c1)CC[C@]1(O)Cc3cc(O)c(O)cc3[C@H]21] |
| 7 | 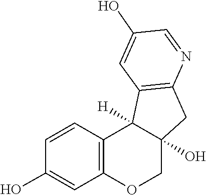 [SMILES: Oc1ccc2c(c1)OC[C@]1(O)Cc3ncc(O)cc3[C@H]21] |
| 8 | 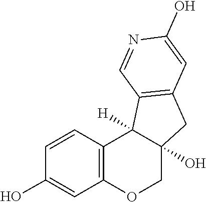 [SMILES: Oc1ccc2c(c1)OC[C@]1(O)Cc3cc(O)ncc3[C@H]21] |
| 8 | 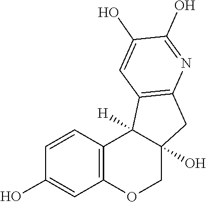 [SMILES: Oc1ccc2c(c1)OC[C@]1(O)Cc3nc(O)c(O)cc3[C@H]21] |
| 10 | 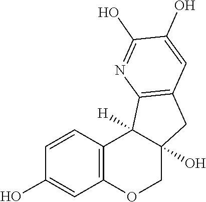 [SMILES: Oc1ccc2c(c1)OC[C@]1(O)Cc3cc(O)c(O)nc3[C@H]21] |
| 11 | 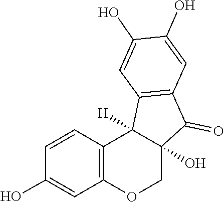 [SMILES: O=C1c2cc(O)c(O)cc2[C@@H]2c3ccc(O)cc3OC[C@]12O] |
| 12 | 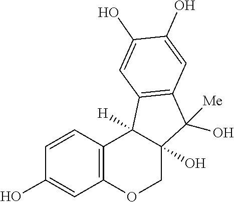 [SMILES: CC1(O)c2cc(O)c(O)cc2[C@@H]2c3ccc(O)cc3OC[C@@]21O] |
| 13 | 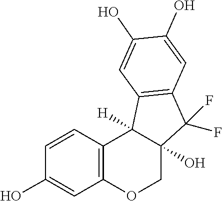 [SMILES: Oc1ccc2c(c1)OC[C@@]1(O)[C@@H]2c2cc(O)c(O)cc2C1(F)F] |
| 14 | 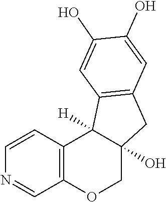 [SMILES: Oc1cc2c(cc1O)[C@@H]1c3ccncc3OC[C@]1(O)C2] |
| 15 | 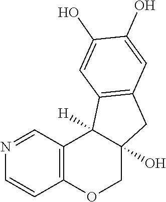 [SMILES: Oc1cc2c(cc1O)[C@@H]1c3cnccc3OC[C@]1(O)C2] |
| 16 | 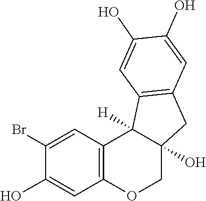 [SMILES: Oc1cc2c(cc1O)[C@@H]1c3cc(Br)c(O)cc3OC[C@]1(O)C2] |
| 17 | 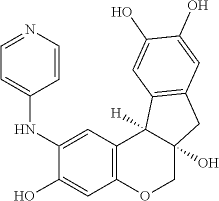 [SMILES: Oc1cc2c(cc1O)[C@@H]1c3cc(Nc4ccncc4)c(O)cc3OC[C@]1(O)C2] |
| 18 | 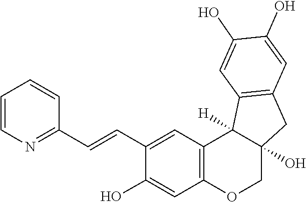 [SMILES: Oc1cc2c(cc1O)[C@@H]1c3cc(/C=C/c4ccccn4)c(O)cc3OC[C@]1(O)C2] |
| 19 | 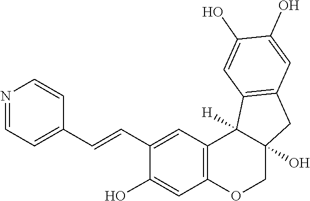 [SMILES: Oc1cc2c(cc1O)[C@@H]1c3cc(/C=C/c4ccncc4)c(O)cc3OC[C@]1(O)C2] |
| 20 | 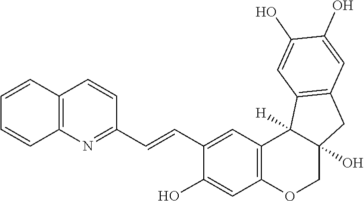 [SMILES: Oc1cc2c(cc1O)[C@@H]1c3cc(/C=C/c4ccc5ccccc5n4)c(O)cc3OC[C@]1(O)C2] |
| 21 | 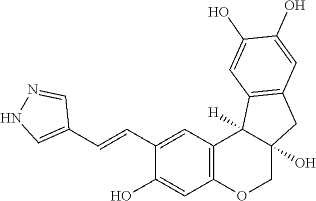 [SMILES: Oc1cc2c(cc1O)[C@@H]1c3cc(/C=C/c4cn[nH]c4)c(O)cc3OC[C@]1(O)C2] |
| 22 | 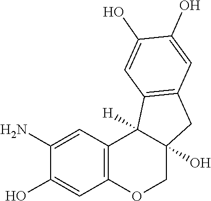 [SMILES: Nc1cc2c(cc1O)OC[C@]1(O)Cc3cc(O)c(O)cc3[C@H]21] |
| 23 | 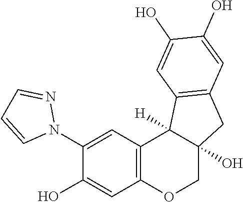 [SMILES: Oc1cc2c(cc1O)[C@@H]1c3cc(-n4cccn4)c(O)cc3OC[C@]1(O)C2] |
| 24 | 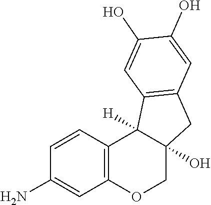 [SMILES: Nc1ccc2c(c1)OC[C@]1(O)Cc3cc(O)c(O)cc3[C@H]21] |
| 25 | 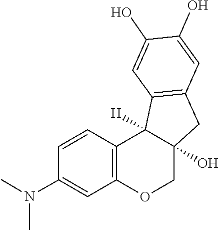 [SMILES: CN(C)c1ccc2c(c1)OC[C@]1(O)Cc3cc(O)c(O)cc3[C@H]21] |
| 26 | 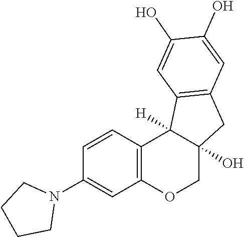 [SMILES: Oc1cc2c(cc1O)[C@@H]1c3ccc(N4CCCC4)cc3OC[C@]1(O)C2] |
| 27 | 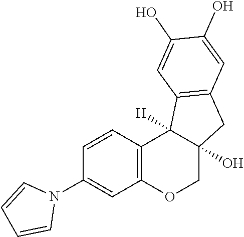 [SMILES: Oc1cc2c(cc1O)[C@@H]1c3ccc(-n4cccc4)cc3OC[C@]1(O)C2] |
| 28 | 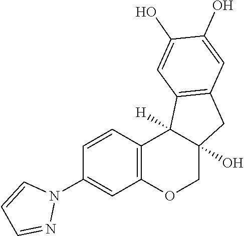 [SMILES: Oc1cc2c(cc1O)[C@@H]1c3ccc(-n4cccn4)cc3OC[C@]1(O)C2] |
| 29 | 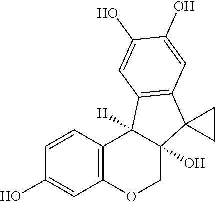 [SMILES: Oc1ccc2c(c1)OC[C@@]1(O)[C@@H]2c2cc(O)c(O)cc2C12CC2] |
| 30 | 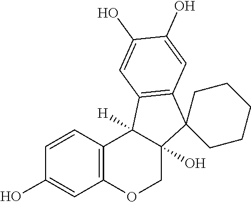 [SMILES: Oc1ccc2c(c1)OC[C@@]1(O)[C@@H]2c2cc(O)c(O)cc2C12CCCCC2] |
| 31 | 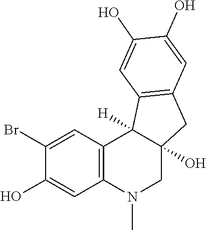 [SMILES: CN1C[C@]2(O)Cc3cc(O)c(O)cc3[C@@H]2c2cc(Br)c(O)cc21] |
| 32 | 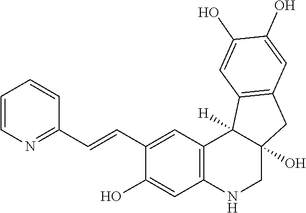 [SMILES: Oc1cc2c(cc1O)[C@@H]1c3cc(/C=C/c4ccccn4)c(O)cc3NC[C@]1(O)C2] |
| 33 | 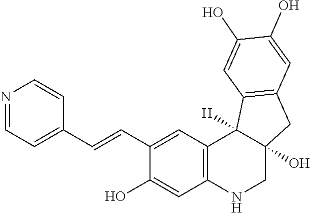 [SMILES: Oc1cc2c(cc1O)[C@@H]1c3cc(/C=C/c4ccncc4)c(O)cc3NC[C@]1(O)C2] |
| 34 | 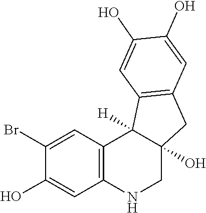 [SMILES: Oc1cc2c(cc1O)[C@@H]1c3cc(Br)c(O)cc3NC[C@]1(O)C2] |

TABLE 2
Results of Detection of DNA, RNA and Protein bindings using the fused tetracyclic compounds

| Compounds | | DNA | RNA | Protein |
|---|---|---|---|---|
| 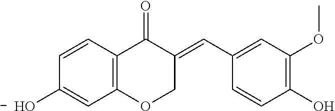 [SMILES: COc1cc(/C=C2\COc3cc([O-])ccc3C2=O)ccc1O] | 007 | yes | Yes | |
| 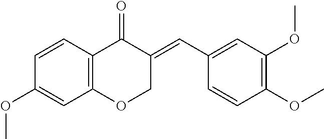 [SMILES: COc1ccc2c(c1)OC/C(=C\c1ccc(OC)c(OC)c1)C2=O] | 009 | | yes | |
| 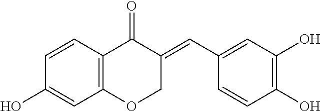 [SMILES: O=C1/C(=C/c2ccc(O)c(O)c2)COc2cc(O)ccc21] | 015 | yes | yes | |
| 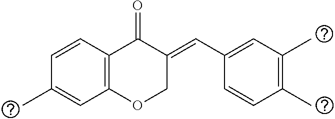 | 016 | yes | | |
| 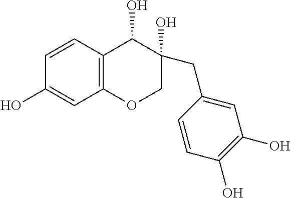 [SMILES: Oc1ccc2c(c1)OC[C@](O)(Cc1ccc(O)c(O)c1)[C@H]2O] | 017 | yes | yes | |
| 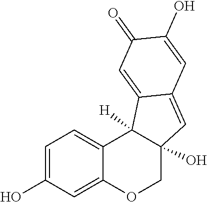 [SMILES: O=C1C=C2C(=C[C@@]3(O)COc4cc(O)ccc4[C@@H]23)C=C1O] | 019 | yes | | |
| 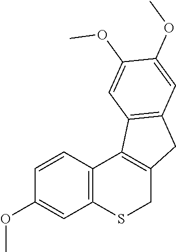 [SMILES: COc1ccc2c(c1)SCC1=C2c2cc(OC)c(OC)cc2C1] | 021 | | yes | |
| 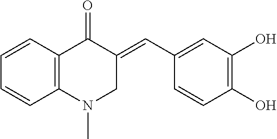 [SMILES: CN1C/C(=C\c2ccc(O)c(O)c2)C(=O)c2ccccc21] | 024 | | | |
| 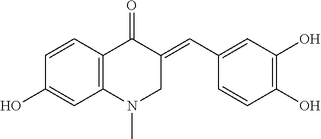 [SMILES: CN1C/C(=C\c2ccc(O)c(O)c2)C(=O)c2ccc(O)cc21] | 025 | | yes | |
| 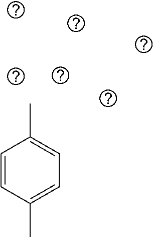 | 026 | yes | yes | |
| 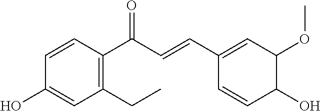 [SMILES: CCc1cc(O)ccc1C(=O)/C=C/C1=CC(OC)C(O)C=C1] | 031 | | yes | |
| 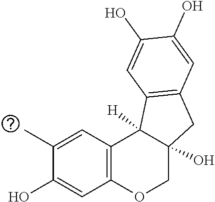 [SMILES: Cc1cc2c(cc1O)OC[C@]1(O)Cc3cc(O)c(O)cc3[C@H]21] | 032 | yes | | |
| 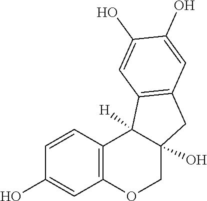 [SMILES: Oc1ccc2c(c1)OC[C@]1(O)Cc3cc(O)c(O)cc3[C@H]21] | 033 | yes | yes | |
| 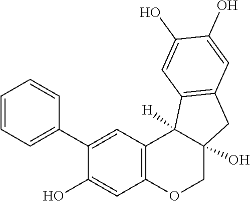 [SMILES: Oc1cc2c(cc1O)[C@@H]1c3cc(-c4ccccc4)c(O)cc3OC[C@]1(O)C2] | 035 | | yes | yes |
| 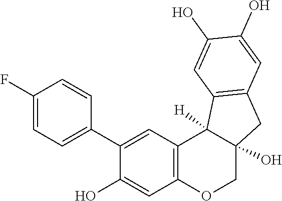 [SMILES: Oc1cc2c(cc1O)[C@@H]1c3cc(-c4ccc(F)cc4)c(O)cc3OC[C@]1(O)C2] | 037 | yes | yes | yes |
| 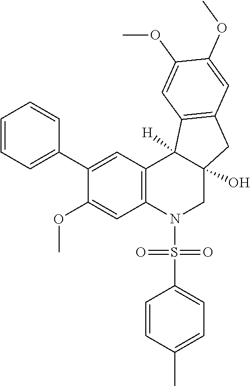 [SMILES: COc1cc2c(cc1OC)[C@@H]1c3cc(-c4ccccc4)c(OC)cc3N(S(=O)(=O)c3ccc(C)cc3)C[C@]1(O)C2] | 038 | | yes | |
| 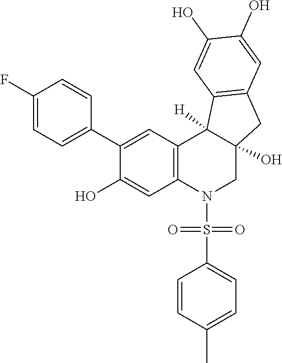 [SMILES: Cc1ccc(S(=O)(=O)N2C[C@]3(O)Cc4cc(O)c(O)cc4[C@@H]3c3cc(-c4ccc(F)cc4)c(O)cc32)cc1] | 039 | yes | | |

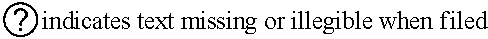

### Brazilin Synthesis: —

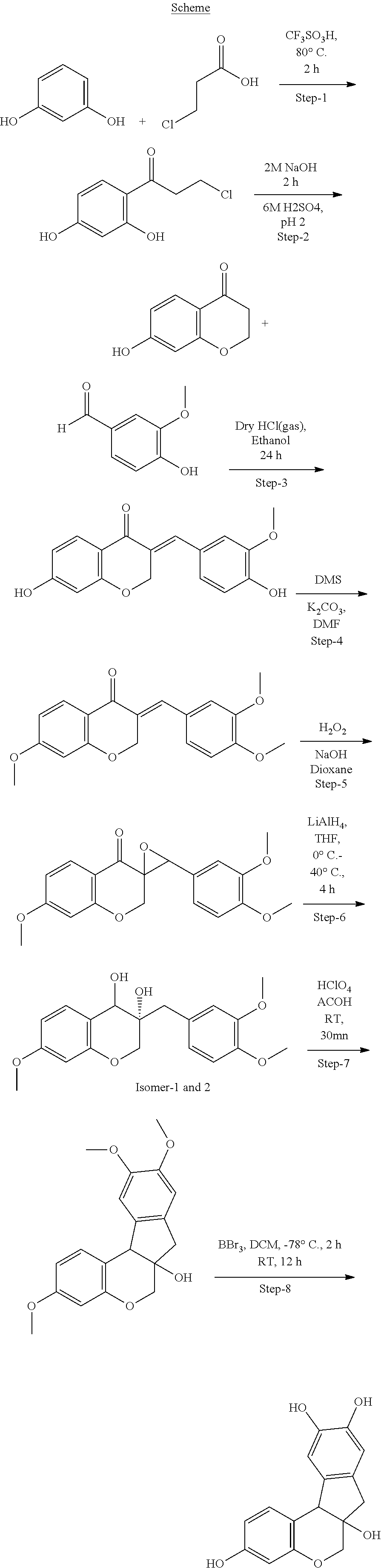

### Step-1: Synthesis of 3-chloro-1-(2,4-dihydroxyphenyl)propan-1-one

To a stirred solution of resorcinol (10 g, 90.8 mmol) and chloropropionic acid (11.8 g, 108.9 mmol) in 250 ml RB flask, added trifluoromethane sulphonic acid (30 ml) slowly with constant stirring. The reaction mixture was heated to 80° C. for 2 h. Further, the reaction mixture was cooled to room temperature, and poured to cold water (500 ml), and extracted with chloroform (3×100). The combined organic layers were washed with brine (100 mL), dried over Na₂SO₄, and concentrated under reduced pressure. Yield 9.1 g, (50.1%). Orange solid.

### Step-2: Synthesis of 7-hydroxy-2,3-dihydro-4H-chromen-4-one

3-chloro-1-(2,4-dihydroxyphenyl)propan-1-one (5.2 g, 31.7 mmol) in 500 ml RB flask, added NaOH solution (2M, 250 ml) slowly with constant stirring at 5° C. The reaction mixture was stirred at RT for 2 h. After completion of the reaction, reaction mixture was acidified with H₂SO₄ (6M) until pH-2. Further reaction mixture was diluted in water (100 ml) extracted with diethyl ether. Combined organic layer was dried over Na₂SO₄, and concentrated under reduced pressure. Yield 3.2 g, (83%) Yellow solid.

### Step-3: Synthesis of (3E)-7-hydroxy-3-(4-hydroxy-3-methoxybenzylidene)-2,3-dihydro-4H-chromen-4-one

To a stirred solution of 7-hydroxy-2,3-dihydro-4H-chromen-4-one (10.5 g, 64.0 mmol) and vaniline (11.6 g, 76.8 mmol) in ethanol (80 ml). The solution was saturated with HCl gas while its temperature was maintained at 30-40° C. with cooling. The reaction mixture was kept at room temperature overnight, and then reaction mixture was poured into cold water (500 ml). The crude mixture was filtered and dried. Crude compound was purified by column chromatography using (40% EtOAc/Hexane). Yield 14 g (yield 75%) red solid.

### Step-4: Synthesis of (3E)-3-(3,4-dimethoxybenzylidene)-7-methoxy-2,3-dihydro-4H-chromen-4-one

To a stirred solution of (3E)-7-hydroxy-3-(4-hydroxy-3-methoxybenzylidene)-2,3-dihydro-4H-chromen-4-one (6 g, 20.1 mmol) in Acetone (100 ml) was added dimethylsulphate (12.6 g, 100.0 mmol) and K₂CO₃ (8.3 g, 60.3 mmol) at RT. Further, the reaction mixture was heated at 60° C. for 12 h. After completion of the reaction, solid K₂CO₃ was filtered through celite bed, solvent was removed by Rota vapor; crude compound was dissolved in water and extracted with ethyl acetate (2×100 mL). The combined organic layers were washed with brine (100 mL), dried over Na₂SO₄, and concentrated under reduced pressure. The resulting brown oil was purified by flash column chromatography (EtOAc:Hexane 3:7) to afford the title compound. Yellow solid 6.2 g (Yield 94%).

### Step-5: Synthesis of 3′-(3,4-dimethoxyphenyl)-7-methoxy-4H-spiro[chromene-3,2′-oxiran]-4-one

To a stirred solution of (3E)-3-(3,4-dimethoxybenzylidene)-7-methoxy-2,3-dihydro-4H-chromen-4-one (5 g, 15.3 mmol) in 1,4Dioxane (150 ml) was added 30% H₂O₂ in water (50 ml) at 0° C. Further added 5% NaOH in water (10 ml), and the resulting mixture was stirred at rt for 8 h. Reaction mixture was diluted with 100 ml water, solid precipitate was filtered and dried by high vacuum to afford the desired product 4.2 g (80%) as a white color solid.

### Step-6: Synthesis of 3-(3,4-dimethoxybenzyl)-7-methoxy-3,4-dihydro-2H-chromene-3,4-diol

To a stirred suspension of 3′-(3,4-dimethoxyphenyl)-7-methoxy-4H-spiro[chromene-3,2′-oxiran]-4-one (3.5 g, 10.2 mmol) in THE (100 ml), added LAH (1.1 g, 29.6 mol) at 0° C. under nitrogen atmosphere. Further, the resulting mixture was stirred at 40° C. for 4 h. Reaction was monitored by TLC, after completion the reaction was cooled to 0° C., quenched with saturated ammonium chloride (20 ml), added water (100 ml), extracted with ethyl acetate (250 ml×3). Organic layer was washed with a saturated brine solution (100 ml). Organic layer was evaporated under reducing pressure. Crude was taken as such without further purification. Crude 3.3 g brown liquid.

### (OR)

### Isomers Separation Method:—

Crude was purified by the Comb flash chromatography by eluting with 20% Acetone in hexane. Both isomers will be separated by this method.

### Step-7: Synthesis of Cyclized Compound

To a stirred solution of 3-(3,4-dimethoxybenzyl)-7-methoxy-3,4-dihydro-2H-chromene-3,4-diol (1.80 g, 5.84 mmol) in acetic acid (50 ml,) was added HClO₄ (0.5 ml) at RT for 30 min. Reaction was monitored by TLC, after completion of the reaction, reaction mixture was cooled to room temperature, solvent was evaporated and crude compound was diluted with saturated sodium bicarbonate solution and extracted in ethyl acetate. The combined organic layers were washed with brine (100 mL), dried over Na₂SO₄, and concentrated under reduced pressure. The resulting solid was purified by flash column chromatography (10% EA in DCM) to afford the title compound 1.4 g (Yield 82%).

### Step-8: Synthesis of Cyclized Compound Demethylated: Isomer-2

To a stirred solution of cyclized compound (0.350 g, 1.067 mmol) in DCM (50 ml,) was added Boron Tribromide (1M in DCM) (10.6 ml, 10.67 mmol) at −78° C., and the resulting mixture was stirred at rt for 16 h. After completion the reaction is monitored by TLC, then quenched slowly with water at 0° C. Extracted with Ethyl acetate (100 ml×3). Crude was purified by prep TLC. The Desired compound isolated 150 mg as pink color solid. (Yield 49%).

¹HNMR (400 MHz, Cd₃OD): 7.17 (d, J=8.4 Hz, 1H), 6.699 (s, 1H), 6.592 (s, 1H), 6.47-6.44 (m, 1H), 6.286-6.281 (m, 1H), 3.955-3.907 (m, 2 h), 3.699-3.670 (m, 1H), 3.03-2.99 (m, 1H) 2.78-2.74 (m, 1H). HPLC: −99%, LCMS: 287(M+1).

### Step-8: Synthesis of Cyclized Compound Demethylated: Isomer-1

To a stirred solution of cyclized compound (0.700 g, 2.134 mmol) in DCM (100 ml,) was added Boron Tribromide (1M in DCM) (21.34 ml, 21.341 mmol) at −78° C., and the resulting mixture was stirred at rt for 16 h. After completion the reaction was monitored by TLC, then quenched slowly with water at 0° C. Extracted with Ethyl acetate (100 ml×3). Crude was purified by prep TLC. The desired compound isolated 380 mg as yellow color solid. (Yield 49%).

¹HNMR (400 MHz, Cd₃OD): 7.17 (d, J=8.0 Hz, 1H), 6.698 (s, 1H), 6.589 (s, 1H), 6.45 (d, d, J=8.0 Hz, 1H), 6.28-6.279 (m, 1H), 3.954-3.906 (m, 2 h), 3.69-3.66 (m, 1H), 3.03-2.99 (m, 1H) 2.78-2.74 (m, 1H). HPLC: 99%, LCMS: 287(M+1).

### Specifications for Cell-Staining Using Nucleic Acid Staining Agents Contained in the Invention

| | |
|---|---|
| For Use With | Microscopy & Flow Cytometry |
| Cell Permeability | Cell Permeable. |
| | Nucleus Specific |
| Detection Method | Fluorescence |
| Excitation/Emission | 490 nm/530 nm: 488 or FITC channel |
| Product Size | 1 mg/ml |
| Solubility | PBS |
| Shipping & Storage | Room Temperature |
| Temperature | |

TABLE 3
Comparative efficacies of the Presented Invention. Following Table shows the
Comparison of the Presented Invention with commonly available DNA staining agents.

| | Compounds | | | |
|---|---|---|---|---|
| | Of the Instant | | | |
| | Invention | Hoescht | Nucspot | DAPI |
| Photostability | 48 hrs | | 24-72 Hrs | 10 min |
| Speed (Incubation) | 5 mins | 5-30 mins | 10 mins< | 15-30 mins |
| Brightness | ++++ | ++++ | ++++ | +++ |
| Exposure time | | | | |
| live/Fixed cell stain | Live cell stain | Live & fixed cell | Live & Fixed | Live & fixed |
| | | stain | cell stain | cell stain |
| Permeabilization | Cell membrane | Cell membrane | Cell membrane | Cell |
| | Permeable | Permeable | permeable | membrane |
| | | | | permeable but |
| | | | | will damage |
| | | | | the cell |
| Anti fade agents | No antifade | Prolong antifade | verapamil(Toxic) | Slow fade |
| | needed | | | gold |
| Background signal | Faint | background | faint | background |
| | Background | signal is visible | background | signal is |
| | signal | | signal | visible |
| Toxicity | non toxic | Potentially | Low toxicity | Potentially |
| | | carcinogenic & | | carcinogenic |
| | | Mutagenic | | & Mutagenic |
| Wash step | No wash step | Needed | No wash step | Needed |

### Absorbance and Fluorescence Studies

Experimental conditions: Excitation Max: 490/530 nm; Emission Max: 560 nm; Band width: 488-600 nm; make: Alexa Fluor 488 (FOR FLOW CYTOMETRY AND MICROSCOPIC DETERMINATIONS)

### Procedure:

1 mg/ml stock solutions of all compounds the were prepared in DMSO. Few insoluble compounds were then dissolved in nonpolar solvent. 10 ul (1 mg/ml) of each of the compounds were used to do an absorbance scan in the TECAN nano reader and the graphs were plotted. The absorbance peaks for each of the compounds were tabulated and further used as excitation wavelength for fluorescence scans. Initially an absorbance scan was performed to identify the excitation maxima after which the excitation wavelength was used to obtain the fluorescence plots. Once the excitation and emission maxima were known, different concentrations of the compounds were used starting from 10 ng to saturating concentrations to obtain fluorescent spectra. Similarly, a fixed concentration of the said compound was used to which an increasing concentration of DNA/RNA was added until saturation to find the nucleic acid binding properties of the dyes and compounds. In addition, a fixed concentration of DNA/RNA was used, to which increasing concentrations of the compounds were added until saturation. The data obtained was plotted and used for calculating binding constants and determining the binding ability of the dyes and compounds.

### Quantum Yield Determination

The fluorescence quantum yield ((DF) is the ratio of photons absorbed to photons emitted through fluorescence. In other words, the quantum yield gives the probability of the excited state being deactivated by fluorescence rather than by another, non-radiative mechanism. Standard samples were chosen to ensure that they absorb at the excitation wavelength of choice for the test sample, and, if possible, emit in a similar region to the test sample. Fluorescein dissolved in 0.1M NaOH with a quantum yield of 0.79 and emission range of 500-600 nm was chosen as the standard for quantum yield determination of the present invention.

Procedure: The following steps formed the procedure for recording fluorescence and absorbance and excitation spectra to obtain quantum yield determination.
1. Record the UV-vis absorbance spectrum of the solvent background for the chosen sample. Note down the absorbance at the excitation wavelength to be used.
2. Record the fluorescence spectrum of the same solution in the 10 mm fluorescence cuvette. Calculate and note down the integrated fluorescence intensity (the area of the fluorescence spectrum) from the fully corrected fluorescence spectrum.
3. Repeat steps 1, and 2. for five solutions with increasing concentrations of the chosen sample. (There will be six solutions in all, corresponding to absorbances at the excitation wavelength of ˜0/solvent blank, 0.02, 0.04, 0.06, 0.08 and 0.10.).
4. Plot a graph of integrated fluorescence intensity vs absorbance. The result should be a straight line with gradient m, and intercept=0.
5. Repeat steps 1 to 4 for the remaining samples.

### Calculation of Fluorescence Quantum Yield:

The gradients of the graphs obtained are proportional to the quantum yield of the different samples. Absolute values are calculated using the standard samples which have a fixed and known fluorescence quantum yield value, according to the following equation:

$$i \cdot {Φ}_{X} = {Φ}_{ST} \{ {Grad}_{X} \} \{ {η}_{X}^{2} \}$$

$$\{ {Grad}_{ST} \} \{ {\_η}_{ST}^{2} \}$$

Where the subscripts ST and X denote standard and test respectively, Φ is the fluorescence quantum yield, Grad the gradient from the plot of integrated fluorescence intensity vs absorbance, and η the refractive index of the solvent.

### Procedure for Performing Agarose Gel Electrophoresis:

Agarose gel electrophoresis is a type of gel electrophoresis used to separate mixed populations of macromolecules such as DNA, RNA or proteins in a matrix of agarose. It is used to determine the approximate length of a DNA, RNA or PCR fragments by running them on an agarose gel alongside a DNA ladder.

Method: 12 RNA samples with control RNA and DNA sample with one control DNA were run on the agarose gel alongside the DNA ladder and the bands were visualized under UV transilluminator.

### Pouring a Standard 1% Agarose Gel and Measuring the Agarose According to the Casting Tray:

Mix agarose powder with 1×TAE in a microwavable flask (Volume of TAE depends on the agarose weight) Microwave for 1-3 min until the agarose is completely dissolved. Let agarose solution cool down to about 50° C. (about when you can comfortably keep your hand on the flask, about 5 mins). Pour the agarose into a gel tray with the well comb in place. Place newly poured gel at 4° C. for 10-15 mins OR let it at room temperature for 20-30 mins, until it has completely solidified. Once solidified, place the agarose gel into the gel box (electrophoresis unit). Fill the gel box with 1×TAE (or TBE) until the gel is covered. Add a loading buffer to each of the DNA samples. Carefully load a molecular weight ladder into the first lane of the gel. Carefully load samples into the additional wells of the gel. Run the gel at 80-150 V until the dye line is approximately 75-80% of the way down the gel. A typical run time is about 1-1.5 hours, depending on the gel concentration and voltage Turn OFF power, disconnect the electrodes from the power source, and then carefully remove the gel from the gel box Post staining, visualize the DNA fragments using the UV transilluminator. The fragments of DNA are usually referred to as ‘bands’ due to their appearance on the gel. The concentration of the dyes and compounds used are in between 10-1 ug, optimal is 50 ng to stain nucleic acids.

Result: The staining agent was able to stain living cells, nucleus, plant cell walls, and yeast cells on par with DAPI.

### Procedure for 50×TAE and 5×TBE Buffer Preparation:

50×TAE and 5×TBE Buffers are prepared as per the concentrations of Tris base, Glacial acetic acid, boric acid and EDTA given in the tables 4 & 5 using Milli Q water and made up to a volume of 1000 ml. using. 1× buffers are prepared from the respective 5×TAE and TBE buffers by diluting them to the volumes needed using milli Q water.

TABLE 4
50× TAE buffer preparation

| Chemical | Mol wt. | Conc | Vol 1000 ml | |
|---|---|---|---|---|
| Tris base | 121.14 | 2M | 242 | g |
| Glacial Acetic Acid | 60.05 | 1M | 57.1 | ml |
| EDTA | 372.24 | 50 mM | 100 | ml |

TABLE 5
5× TBE Buffer preparation

| Chemical | Mol wt. | Conc | Vol 1000 ml | |
|---|---|---|---|---|
| Tris base | 121.14 | 0.45M | 54 | g |
| Boric acid | 61.83 | 0.45M | 27.5 | |
| EDTA | 372.24 | 0.01M | 20 | ml |

### Experimental Embodiments

The present invention is further elaborated with help of the following Figures. Followings are the detailed description of the accompanying figures.

### RNA Binding Studies:

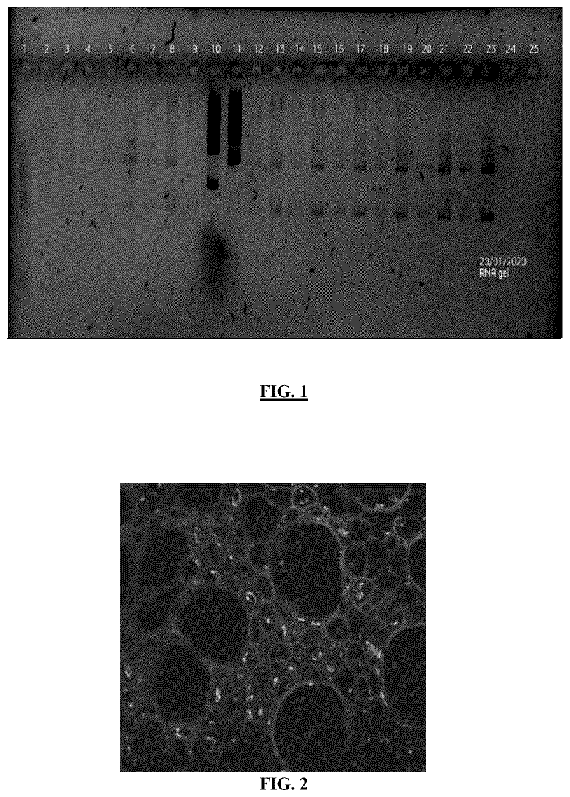

FIG. 1: Illustrates agarose gel shows 50 and 100 ng concentrations of RNA stained with different compounds respectively. Lane1—1 kbp, Lane2—50 ng RNA 007, Lane3—100 ng RNA 007, Lane4—50 ng RNA 009, Lane5—100 ng RNA 009, Lane6—100 ng RNA 17, Lane7—50 ng RNA 17, Lane8—100 ng RNA 21, Lane9—50 ng RNA 21, Lane10—50 ng RNA 25, Lane11—100 ng RNA 25, Lane12—50 ng RNA 26, Lane13—100 ng RNA 26, Lane14—50 ng RNA 31, Lane15—100 ng RNA 31, Lane16—50 ng RNA 33, Lane17—100 ng RNA 33, Lane18—50 ng RNA 35, Lane19—100 ng RNA 35, Lane20—50 ng RNA 37, Lane21—100 ng RNA 37, Lane22—50 ng RNA 38, Lane23—100 ng RNA 38, Lane24—50 ng RNA 003 and Lane25—100 ng RNA 003.

### Confocal/Fluorescence Studies;

FIG. 2: Illustrates transverse section of plant cell stained with one of the compounds viewed under Olympus confocal microscope magnification, 20×.

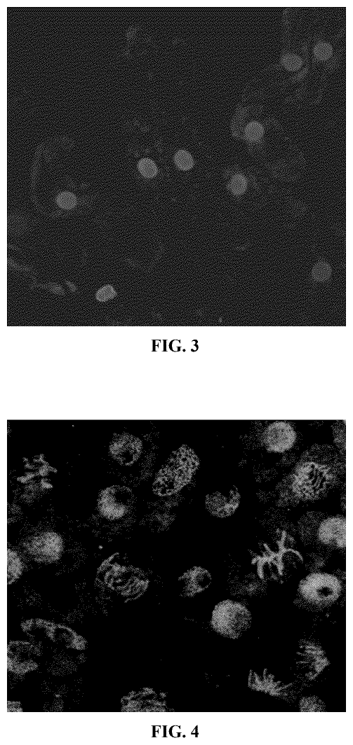

FIG. 3: Illustrates Buccal cell nucleus stained with one of the compounds viewed under fluorescent microscope, 20× magnification.

FIG. 4: Illustrates cell division stages stained with one of the compounds and viewed under a confocal microscope, 20× magnification.

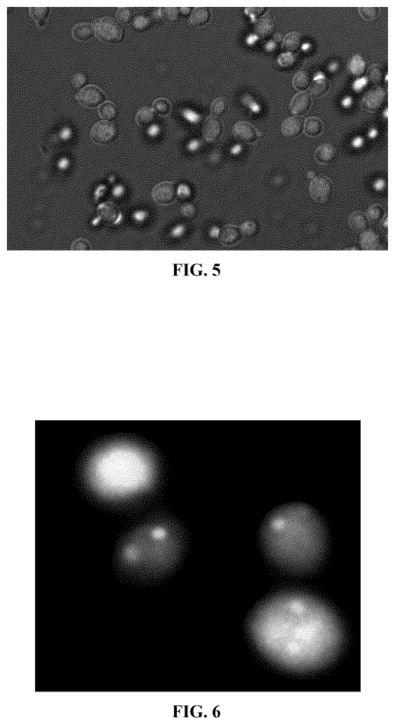

FIG. 5: Illustrating that the compound stains only live yeast cells and not dead cells. The yeast cells were stained without permeabilization and observed under a confocal microscope, 20×.

FIG. 6: Illustrates yeast nuclei clearly stained and viewed under apotome, 100× magnification.

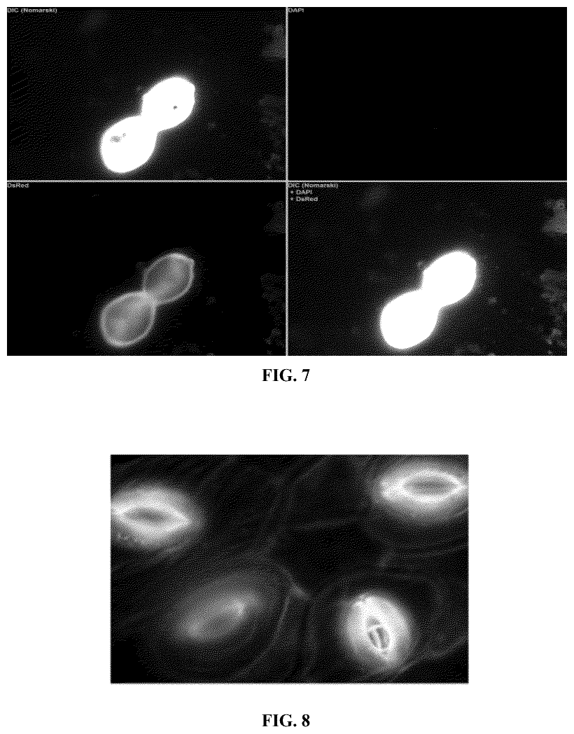

FIG. 7: Illustrates plant pollen stained with compound, 40× magnification.

FIG. 8: Illustrates plant stomata stained, 40× magnification.

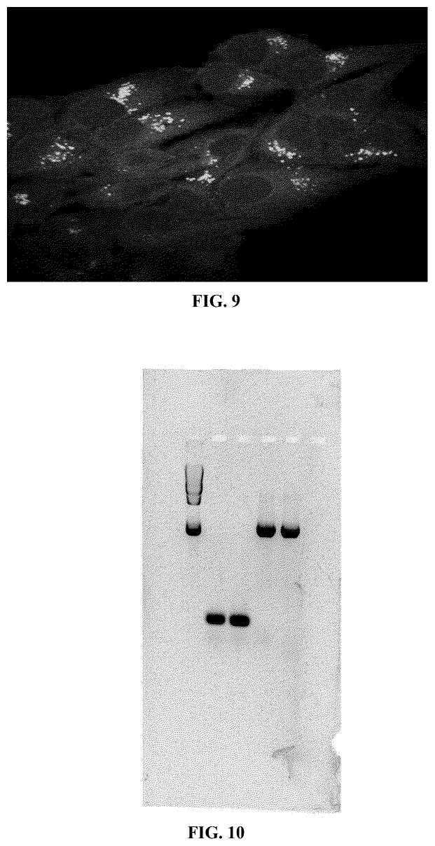

FIG. 9: Illustrates HeLa cells stained with compound and observed under a confocal microscope, 40× magnification.

### DNA Binding Studies:

FIG. 10: Illustrates PCR products stained and observed in Biorad XRS gel doc system. Lane 1—1 kb ladder, Lane 2 to 5—PCR products.

FIG. 11: Illustrates Plasmid DNA stained with compound and viewed under gel doc system. Lane 1—100 bp marker, Lane 2 to 6—50 to 200 ng of plasmid DNA.

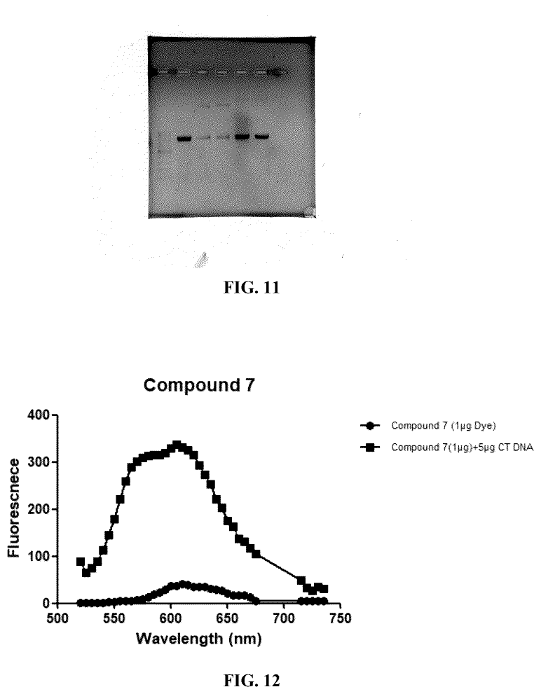

FIG. 12: Illustrates Compound 7 shows 25 fold increase in fluorescence upon binding with Calf Thymus DNA (CT-DNA).

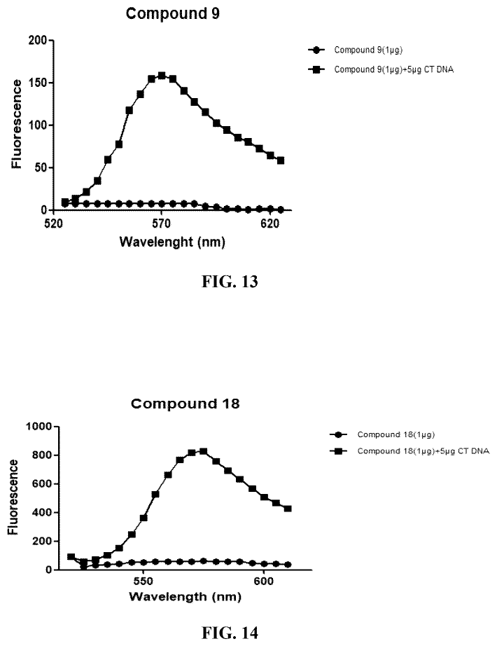

FIG. 13: Illustrates Compound 9 shows 15 fold increase in fluorescence upon binding with Calf Thymus DNA (CT-DNA).

FIG. 14: Illustrates Compound 18 shows 70 fold increase in fluorescence upon binding with Calf Thymus DNA (CT-DNA).

FIG. 15: Illustrates Compound 16 shows 5 fold increase in fluorescence upon binding with Calf Thymus DNA (CT-DNA).

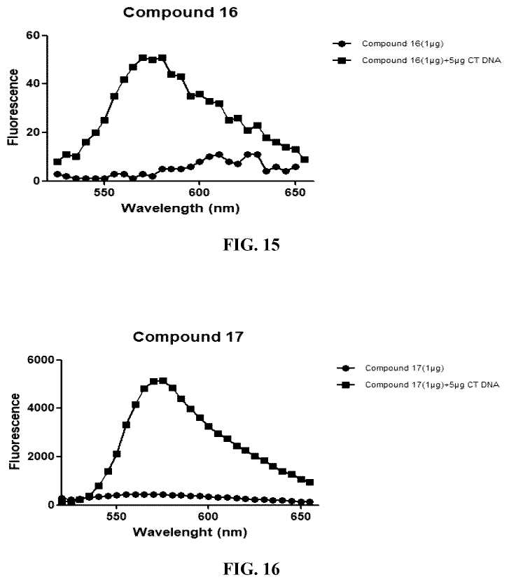

FIG. 16: Illustrates Compound 17 shows >1000 fold increase in fluorescence upon binding with Calf Thymus DNA (CT-DNA).

FIG. 17: Illustrates Compound 19 shows >1000 fold increase in fluorescence upon binding with Calf Thymus DNA (CT-DNA).

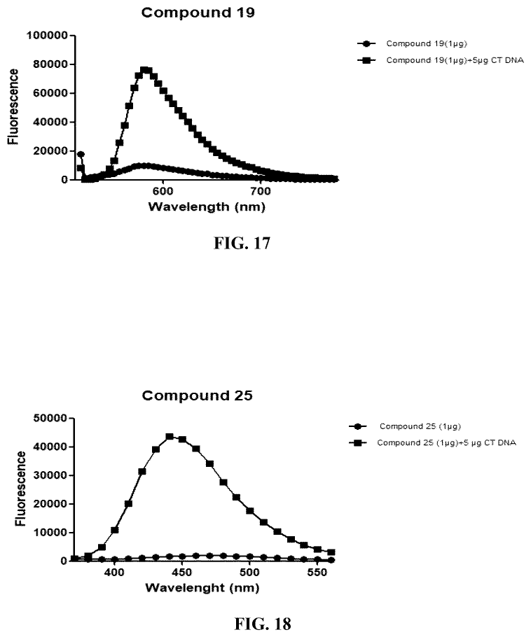

FIG. 18: Illustrates Compound 25 shows >1000 fold increase in fluorescence upon binding with Calf Thymus DNA (CT-DNA).

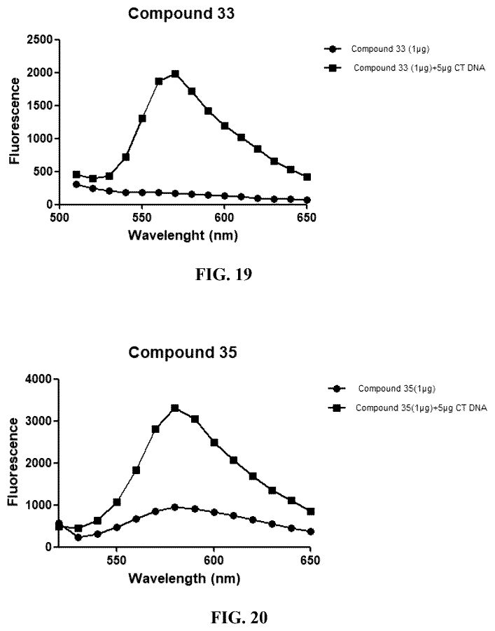

FIG. 19: Illustrates Compound 33 shows >1000 fold increase in fluorescence upon binding with Calf Thymus DNA (CT-DNA).

FIG. 20: Illustrates Compound 35 shows >1000 fold increase in fluorescence upon binding with Calf Thymus DNA (CT-DNA).

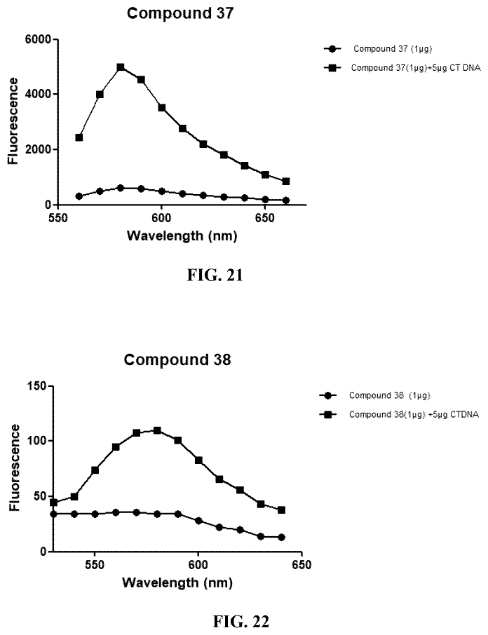

FIG. 21: Illustrates Compound 37 shows >1000 fold increase in fluorescence upon binding with Calf Thymus DNA (CT-DNA).

FIG. 22: Illustrates Compound 38 shows 2 fold increase in fluorescence upon binding with Calf Thymus DNA (CT-DNA).

TABLE 6
Excitation Maxima, Emission maxima and Emission
maxima with DNA obtained with the fused tetracyclic,
heterocyclic compounds of formula (I).

| | Fused tetracyclic | | | |
|---|---|---|---|---|
| | heterocyclic | | | Emission |
| Sl. | compounds [symbol] | Excitation | Emission | maxim [symbol] |
| No | formula (I) | Maxima | maxima | with DNA |
| 1 | 15 | 490 | 570 | 610 |
| 2 | 16 | 490 | 610 | 610 |
| 3 | 19 | 490 | 570 | 570 |
| 5 | 26 | 490 | 580 | 590 |
| 6 | 31 | 490 | 610 | 610 |
| 7 | 32 | 490 | 570 | 570 |
| 8 | 35 | 490 | 580 | 580 |

[symbol]

indicates data missing or illegible when filed

### Procedure to Find Working Concentration of Compound (25) for RNA Detection:

To check the working concentration of compound (25) using different concentrations of RNA, RNA of E. coli BL21 was isolated and diluted to 10 ng/μl from the stock. Using a TBE buffer 1% agarose gel was casted and gel electrophoresis is performed using 10-50 ng concentration of RNA with 5, 10, 20, 40 ng of compound 25. Results obtained showed bands are in all the wells indicating the suitability of the compounds as staining agents to detect RNA.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the invention have been shown and described in detail to illustrate the inventive principles, it will be understood that the invention may be embodied otherwise without departing from such principles.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment.

It should be understood that the figures and/or screen shots illustrated in the attachments highlighting the functionality and advantages of the present invention are presented for example purposes only. The present invention is sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1. Fluorescent nucleic acid staining agents comprising fused tetracyclic, heterocyclic compounds of formula (I), 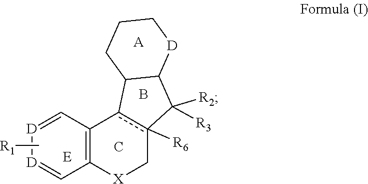
their tautomers, polymorphs, stereoisomers, solvates, wherein,
(a) A is polycyclic heterocyclic ring which is unsaturated or partially unsaturated, optionally having up to two heteroatoms independently selected from O, N or S;
(b) Ring A can be optionally substituted by the atoms or groups comprising hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
(c) D is selected from CR⁴ or N;
(d) X is selected from CR⁴, O, NR⁵ or S;
(e) ring C is saturated or partially unsaturated, when it is saturated R6 can be H or OH whereas in case when ring C is partially unsaturated R6 is absent;
(f) R¹ and R⁴ are selected independently from hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
(g) R¹ and R⁴ can cyclize to form a 4-7 membered ring which can be optionally substituted by hydrogen, halogen, hydroxy, alkoxy, aryloxy, amino, alkylamino, arylamino, hetroarylamino, haloalkyl, alkyl, aryl, arylalkyl, heteroaryl, heteroarylalkyl, cycloalkyl, cycloalkylalkyl, heterocyclyl or heterocyclylalkyl;
(h) R² and R³ are independently selected from hydrogen, alkyl, cycloalkyl alkenyl, alkynyl, alkoxy, acyl, acylamino, optionally R² and R³ can combine to form a 3 to 7 membered ring;
(i) R⁵ sis selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl.

2. The compound of formula (I) as claimed in claim 1 is one of:
(A) 9,10-trimethoxy-5-(p-tolylsulfonyl)-7,11b-dihydro-6H-indeno[2,1-c]quinolin-6a-ol;
(B) 5-(p-tolylsulfonyl)-7,11b-dihydro-6H-indeno[2,1-c]quinoline-3,6a,9,10-tetrol;
(C) (3Z)-3-[(3,4-dimethoxyphenyl)methylene]-7-methoxy-thiochroman-4-one;
(D) 3,9,10-trimethoxy-6,7-dihydroindeno[2,1-c]thiochromene (Isomer-1);
(E) 3,9,10-trimethoxy-6,7-dihydroindeno[2,1-c]thiochromene:—(Isomer-2);
(F) 3,9,10-trimethoxy-6,7-dihydroindeno[2,1-c]thiochromene;
(G) 2-bromo-3,9,10-trimethoxy-7,11b-dihydro-6H-indeno[2,1-c]chromen-6a-ol;
(H) 2-bromo-7,11b-dihydro-6H-indeno[2,1-c]chromene-3,6a,9,10-tetrol;
(I) 3,9,10-trimethoxy-2-phenyl-7,11b-dihydro-6H-indeno[2,1-c]chromen-6a-ol;
(J) 2-phenyl-7,11b-dihydro-6H-indeno[2,1-c]chromene-3,6a,9,10-tetrol;
(K) 2-(4-fluorophenyl)-3,9,10-trimethoxy-7,11b-dihydro-6H-indeno[2,1-c]chromen-6a-ol;
(L) 2-(4-fluorophenyl)-7,11b-dihydro-6H-indeno[2,1-c]chromene-3,6a,9,10-tetrol;
(M) 2-bromo-3,9,10-trimethoxy-5-(p-tolylsulfonyl)-7,11b-dihydro-6H-indeno[2,1-c]quinolin-6a-ol;
(N) 3,9,10-trimethoxy-2-phenyl-5-(p-tolylsulfonyl)-7,11b-dihydro-6H-indeno[2,1-c]quinolin-6a-ol;
(O) 3,9,10-trimethoxy-2-phenyl-5-(p-tolylsulfonyl)-7,11b-dihydro-6H-indeno[2,1-c]quinolin-6a-ol;
(P) 2-(4-fluorophenyl)-3,9,10-trimethoxy-5-(p-tolylsulfonyl)-7,11b-dihydro-6H-indeno[2,1-c]quinolin-6a-ol;
(Q) 2-(4-fluorophenyl)-5-(p-tolylsulfonyl)-7,11b-dihydro-6H-indeno[2,1-c]quinoline-3,6a,9,10-tetrol;
(R) 3-(3,4-dimethoxybenzyl)-7-methoxy-3,4-dihydro-2H-chromene-3,4-diol (Brazilin).

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The compounds of formula (I) as claimed in claim 1 comprising prophetic molecules 1-24 as given in Table 1 with the following structures:

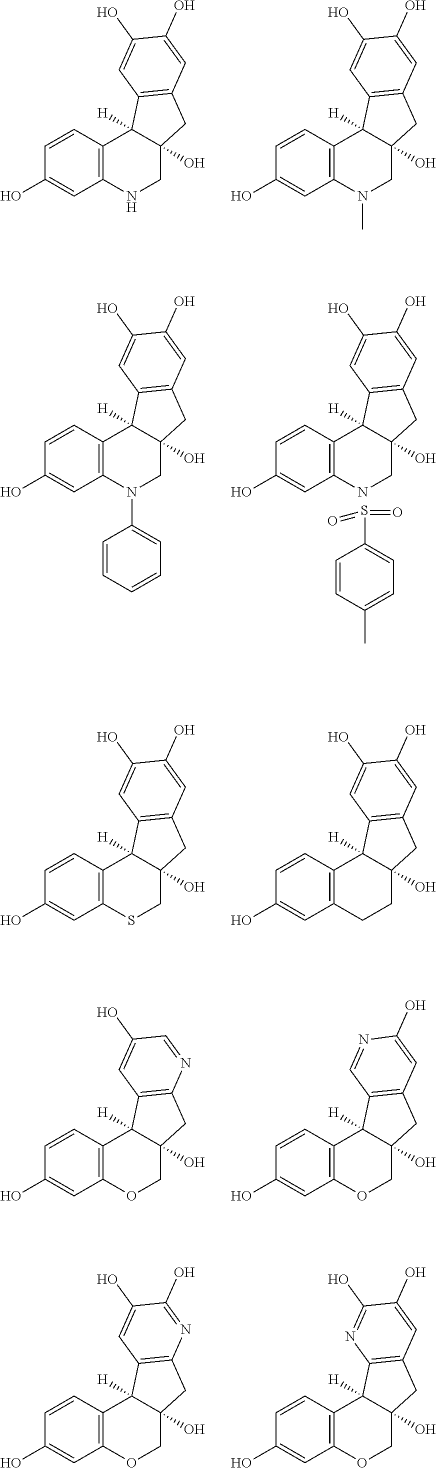

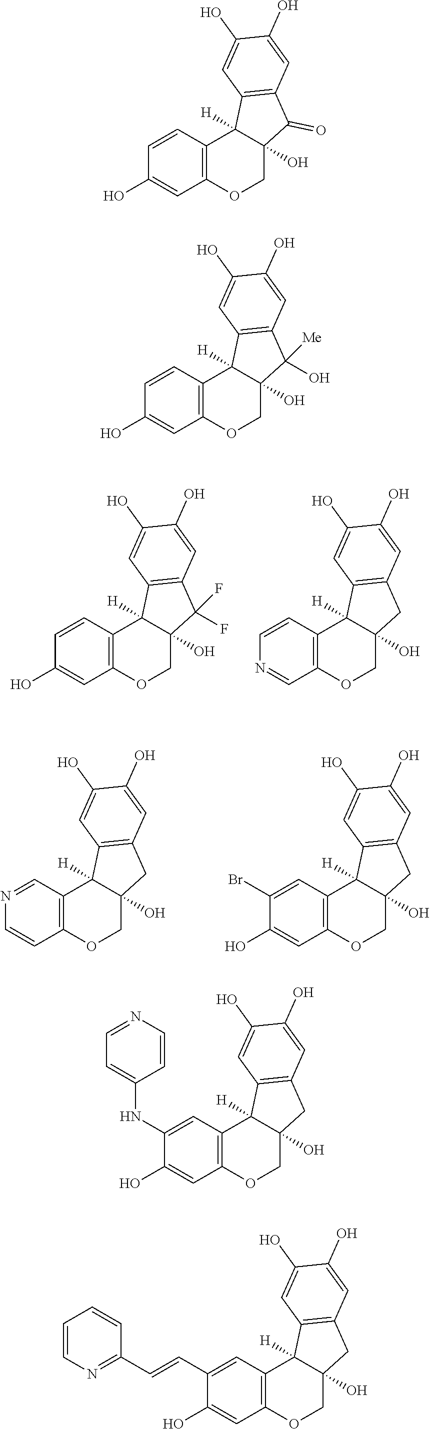

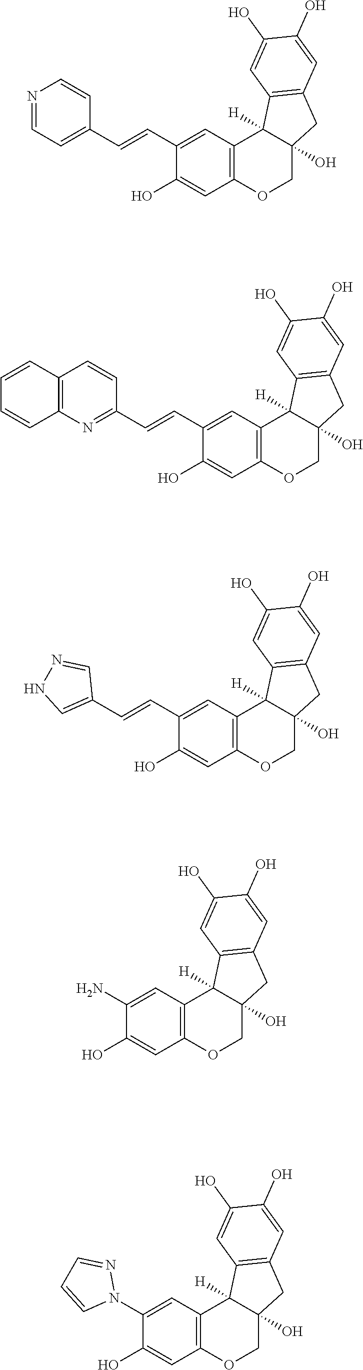 [SMILES: Oc1cc2c(cc1O)[C@@H]1c3cc(/C=C/c4ccncc4)c(O)cc3OC[C@]1(O)C2 ; Oc1cc2c(cc1O)[C@@H]1c3cc(/C=C/c4ccc5ccccc5n4)c(O)cc3OC[C@]1(O)C2 ; Oc1cc2c(cc1O)[C@@H]1c3cc(/C=C/c4cn[nH]c4)c(O)cc3OC[C@]1(O)C2 ; Nc1cc2c(cc1O)OC[C@]1(O)Cc3cc(O)c(O)cc3[C@H]21 ; Oc1cc2c(cc1O)[C@@H]1c3cc(-n4cccn4)c(O)cc3OC[C@]1(O)C2]

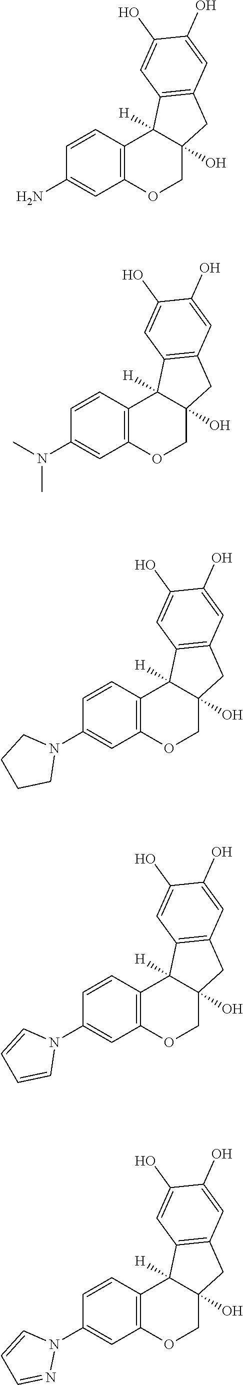 [SMILES: Nc1ccc2c(c1)OC[C@]1(O)Cc3cc(O)c(O)cc3[C@H]21 ; CN(C)c1ccc2c(c1)OC[C@]1(O)Cc3cc(O)c(O)cc3[C@H]21 ; Oc1cc2c(cc1O)[C@@H]1c3ccc(N4CCCC4)cc3OC[C@]1(O)C2 ; Oc1cc2c(cc1O)[C@@H]1c3ccc(-n4cccc4)cc3OC[C@]1(O)C2 ; Oc1cc2c(cc1O)[C@@H]1c3ccc(-n4cccn4)cc3OC[C@]1(O)C2]

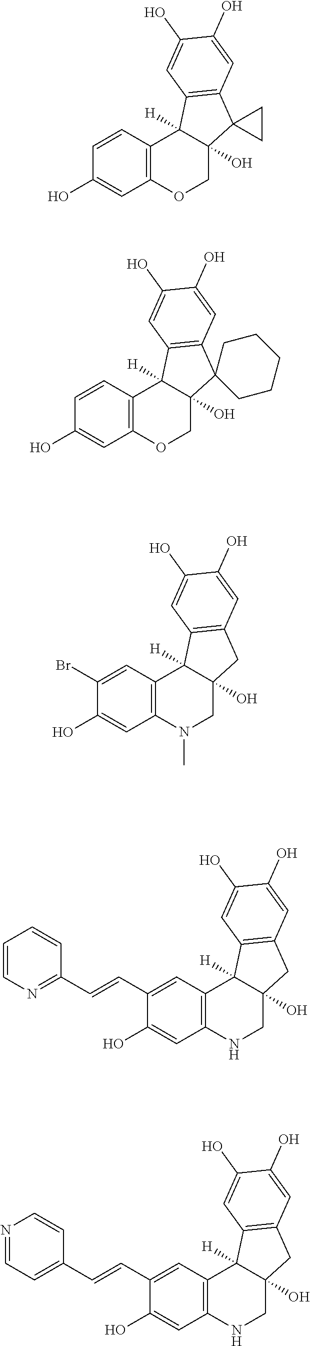 [SMILES: Oc1ccc2c(c1)OC[C@@]1(O)[C@@H]2c2cc(O)c(O)cc2C12CC2 ; Oc1ccc2c(c1)OC[C@@]1(O)[C@@H]2c2cc(O)c(O)cc2C12CCCCC2 ; CN1C[C@]2(O)Cc3cc(O)c(O)cc3[C@@H]2c2cc(Br)c(O)cc21 ; Oc1cc2c(cc1O)[C@@H]1c3cc(/C=C/c4ccccn4)c(O)cc3NC[C@]1(O)C2 ; Oc1cc2c(cc1O)[C@@H]1c3cc(/C=C/c4ccncc4)c(O)cc3NC[C@]1(O)C2]

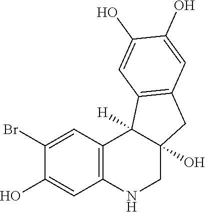 [SMILES: Oc1cc2c(cc1O)[C@@H]1c3cc(Br)c(O)cc3NC[C@]1(O)C2]

21. The fused tetracyclic, heterocyclic fluorescent nucleic acid staining agents as claimed in claim 1 with the structure as given in formula (I) wherein R¹ and R⁴ are additionally selected from a group comprising arylalkyl, heteroaryl, heteroarylalkyl, cycloalkyl, cycloalkylalkyl, heterocyclyl or heterocyclylalkyl, mono- or bi-cyclic non-aromatic carbocyclic group composed of 3 to 10 ring members, and containing one to 3 hetero atoms selected from oxygen, sulphur, SO, SO₂ and nitrogen and further optionally,
(a) it being understood that bicyclic group may be fused or spiro type,
(b) it being possible for the aryl, heteroaryl, cycloalkyl and heterocycloalkyl groups so defined and the alkyl, alkenyl, alkynyl, alkoxy to be substituted by 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆)polyhaloalkyl; trifluoromethoxy; (C₁-C₆)alkylsulphonyl;
halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and additionally salts thereof with a pharmaceutically acceptable acid or base.

22. The compounds of formula (I) as claimed in claim 1 wherein R¹ and R⁴ cyclize to form a 4-7 membered ring which can be additionally substituted by mono- or bi-cyclic non-aromatic carbocyclic group, composed of 3 to 10 ring members, and containing one to 3 hetero atoms selected from oxygen, sulphur, SO, SO₂ and nitrogen and additionally,
(a) it being understood that bicyclic group may be fused or spiro type,
(b) it being possible for the aryl, heteroaryl, cycloalkyl and heterocycloalkyl groups so defined and the alkyl, alkenyl, alkynyl, alkoxy, to be substituted by from 1 to 3 groups selected from: optionally substituted linear or branched (C₁-C₆)alkyl; optionally substituted linear or branched (C₂-C₆)alkenyl group; optionally substituted linear or branched (C₂-C₆)alkynyl group; (C₃-C₆)spiro; optionally substituted linear or branched (C₁-C₆)alkoxy; (C₁-C₆)alkyl-S—; hydroxyl; oxo (or N-oxide where appropriate); nitro; cyano; —COOR′; —OCOR′; —NR′R″; R′CONR″—; NR′R″CO—; linear or branched (C₁-C₆)polyhaloalkyl; trifluoromethoxy; (C₁-C₆)alkylsulphonyl; halogen; optionally substituted aryl; optionally substituted heteroaryl; optionally substituted aryloxy; optionally substituted arylthio; optionally substituted cycloalkyl or optionally substituted heterocycloalkyl, it being understood that R′ and R″ independently of one another represent a hydrogen atom, an optionally substituted linear or branched (C₁-C₆)alkyl group or an aryl-group, their enantiomers and diastereoisomers, and addition salts thereof with a pharmaceutically acceptable acid or base.

23. The compounds of formula (I) as claimed in claim 1 wherein R² and R³ are independently selected from hydrogen, alkyl, cycloalkyl alkenyl, alkynyl, alkoxy, acyl, acylamino, amino or amine group consisting of hydrogen, alkyl, substituted alkyl, aryl, substituted aryl, arylalkyl, substituted aryl alkyl, heteroaryl, and substituted heteroaryl, acyl constituting amino, alkylamino, arylamino, heteroarylamino, acylamino groups, including protonated and quaternized nitrogen comprising the group —NRRR″ and its biologically compatible anionic counterions.

24. The compounds of formula (I) as claimed in claim 1 wherein R⁵ is optionally selected from hydrogen, alkyl, aryl, arylalkyl, heteroaryl, heteroarylalkyl, cycloalkyl, cycloalkylalkyl, heterocyclyl or heterocyclylalkyl or —S(O)₂ alkyl/aryl/heteroaryl or optionally substituted —SO₂ moiety;
wherein, optionally substituted —SO₂ comprising —SO₂-alkyl or aryl or heteroaryl, SR, —SOR, —SO₂R, and R in each of the above groups selected independently from hydrogen, substituted or unsubstituted alkyl, substituted or, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted heteroaryl, substituted heterocyclylalkyl ring, substituted or unsubstituted heteroarylalkyl, or substituted or unsubstituted heterocyclic ring, or any two of R groups joined to form a substituted or unsubstituted saturated or unsaturated 3-10 membered ring, which may optionally include hetero atoms which may be the same or different and are selected from O, NR (wherein R is hydrogen or substituted or unsubstituted alkyl or S).

25. The compounds of formula (I) as claimed in claim 1 are one of:
(A) nucleic acid staining agents to detect nucleic acids from nucleus and non-nucleus cell organelles, from biological samples, scenes of crime, imagery;
(B) fluorescent nucleic acid staining agents to detect proteins, DNA, RNA from cell-lines, cell organelles, mitochondria, fragments, body fluids, tissues, biopsies, swabs, plants, animals, humans, and yeasts;
(C) highly specific, and sensitive fluorescent nucleic acid staining agents, that can detect single-strand, double-strand DNA, RNA, Proteins.

26. (canceled)

27. (canceled)

28. The compounds of formula (I) as claimed in claim 1 as nucleic acid detecting agents used for mass-screening, rapid-screening, monitoring of analytes in case of diseases such as cancer, targeted therapy, fossil-analysis, forensics, and biological profiling.

29. The compounds of formula (I) as claimed in claim 1 and their tautomers, stereoisomers, solvates, polymorphs, formed using generally acceptable inorganic and organic acids.

30. The compounds of formula (I) as claimed in claim 1 finding application as nucleic acid staining agents that are non-intercalating, non-mutagenic, and non-toxic.

31. (canceled)

32. The fluorescent fused tetracyclic, heterocyclic nucleic acid staining compounds of formula (I) as claimed in claim 1 possessing sulphur moieties to facilitate binding to nucleic acids and proteins.

33. A method of detection of nucleic acids by compounds of formula (I) using UV excitation or other spectrofluorimetric methods of analysis.

34. The method of detection of nucleic acids by fluorescent nucleic acid staining by compounds of formula (I) as claimed in claim 33 comprised of a detection apparatus comprising gel documentation system, UV transilluminator or spectrofluorimeter.

35. The method of detection of nucleic acids by fluorescent nucleic acid staining by compounds of formula (I) as claimed in claim 33 comprised of a A-diagnostic kit comprising fluorescent nucleic acid staining agents, fluorescent lamp, uv-spectrofluorimeter and gel documentation wherein the gel is agarose gel.

36. A method of synthesizing the compounds of formula (I) comprising three steps A, B, and C wherein Step A comprises;
i) synthesis of the intermediate IV from aryl-XH with the structure as given,
ii) by Michael addition of aryl-XH and alkyl acrylate/acrylonitrile using bases selected from a group comprising NaH, KOtBu, NaOtBu, K2CO3,
or by alkylation of the compound having the structure of (I) using strong bases to give compound II;
iii) compound II undergoing ester hydrolysis in presence of bases comprising LiOH, NaOH in the solvent media comprising THF, MeOH, water or mixture thereof,
iv) Lewis acid mediated cyclization of compound II leading to compound IV wherein Lewis acids comprising poly phosphoric acid (PPA) or trifluoroacetic acid (TFA) or methanesulfonic acid (MSA), pTSA either in catalytic or stoichiometric amounts are employed for mediating cyclization of compound II to yield compound III and cyclization of II in presence of Lewis acids yielding the intermediate having structure as given in IV;
Step B comprises;
ia) synthesis of compound VI via aldol condensation of corresponding aldehydes and compound IV in the presence of bases comprising NaOH, KOH or acids such as H₂SO₄, triflic acid, acetic acid, HCl or,
ib) by enamine chemistry using a group comprising piperidine/or pyrrolidine/or any secondary aliphatic amine,
ii) epoxidation of exocyclic double bonds of compound VI in presence of oxidizing agents selected from a group comprising H₂O₂, Oxone, TBHP, mCPBA, etc. in the presence/or absence of a base to obtain compound VII,
iii) cyclization of VII to tetracyclic compound IX controlled by acids selected from a group comprising perchloric acid, acetic acid, hydrochloric acid, sulphuric acid, triflic acid, trifluoroacetic acid independently or a mixture thereof in the presence/or absence of solvent such as methanol, ethanol, THF, dioxane, toluene, xylene, etc.
Step C comprises;
synthetic routes for the derivatization of compound XI for obtaining compounds of formula (I) by coupling reactions using catalysts such as palladium in the presence of suitable phosphine ligands of the commercially available palladium phosphine complex as given under wherein;
i) Obtaining compound XII from compound XI using appropriate amine as a coupling partner with compound XI under conditions known in the literature for Buchwald-Hartwig amination which upon deprotection resulting in compound XV,
ii) using compound XI and appropriate catalysts such as boronic acid or boronates under Suzuki coupling conditions for obtaining compound XIII which upon deprotection yielding compound XV,
iii) coupling of compound XI under Heck coupling conditions with appropriate olefins to obtain alkene substituted derivative XIV, which upon further deprotection resulting in compound XVII.

37. The method as claimed in claim 36 yielding compounds of formula (I) with suitable moieties in their structure on derivatization in Step C of the method using suitable derivatizing agents, yielding fused tetracyclic, heterocyclic fluorescent nucleic acid staining agents to detect nucleic acids from analytes.

38. The method as claimed in claim 36 comprises exemplary synthetic routes for obtaining compounds of formula (I) as given in schemes 1-6.

39. The method of synthesis of compounds of formula (I) as claimed in claim 36 includes exemplary synthesis of 3-(3,4-dimethoxybenzyl)-7-methoxy-3,4-dihydro-2H-chromene-3,4-diol (Brazilin) from Resorcinol and chloro-propionic acid.

40. The compounds of formula (I) as claimed in claim 1 comprising molecules according to claims 2, 20 and 39 with the structures given.
